(19) United States
(12) Patent Application Publication
(10) Pub. No.: US 2006/0183780 A1
(43) Pub. Date: Aug. 17, 2006

(54) Pyrazole compounds useful in the treatment of inflammation

(76) Inventors: Anders Hallberg, Uppsala (SE); Wesley Schaal, Uppsala (SE); Mats Larhed, Uppsala (SE); Kristofer Olofsson, Knivsta (SE); Benjamin Pelcman, Stockholm (SE); Andrei Sanin, Stockholm (SE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/547,996

(22) PCT Filed: Mar. 12, 2004
(86) PCT No.: PCT/GB04/01054

§ 371 Date: Nov. 23, 2005

(30) Foreign Application Priority Data
Mar. 14, 2003 (SE) 0300705-1
Jun. 26, 2003 (US) 60482563

(51) Int. Cl. A61K 31/4439; A61K 31/427; A61K 31/4152; C07D 417/02; C07D 403/02

(52) U.S. Cl. 514/341; 514/370; 514/406; 546/275.4; 548/190; 548/374.1

(57) ABSTRACT

There is provided a compound of formula (I), wherein R¹, R², R³, X and Y have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

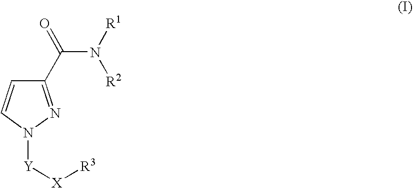

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting of 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

Certain pyrazole compounds that are structurally related to those described herein are commercially available. However, to the knowledge of the applicant, these compounds have never been disclosed in any printed publication and as such have no perceived utility ascribed to them.

Certain pyrazolecarboxylic acid hydrazides, structurally unrelated to the compounds described herein, have been disclosed as anti-inflammatory agents in Tihanyi et al, Eur. J. Med. Chem.—Chim. Ther., 1984, 19, 433 and Goel et al, J. Chem. Inf. Comput. Sci. 1995, 35, 510.

Heterocyclic compounds (including pyrazoles) with anticonvulsant activity have been disclosed in U.S. Pat. No. 5,258,397. Other heterocyclic compounds with antithrombotic activity have been disclosed in WO 02/00651. Neither of these documents discloses or suggests the use of the compounds disclosed therein in the treatment of inflammation.

### DISCLOSURE OF THE INVENTION

According to the invention there is provided a compound of formula I, 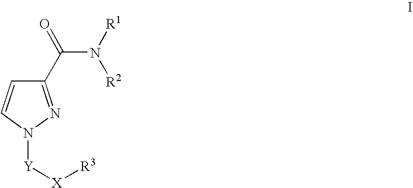
wherein
either
R¹ represents an aryl group or a heteroaryl group, both of which are optionally substituted by one or more substituents selected from G¹ and B¹, which B¹ group may itself be further substituted by one or more substituents selected from G², Z (provided that Z is not directly attached to an aryl or a heteroaryl group) and B² (which B² group is optionally further substituted by one or more substituents selected from G³, B³ and Z, provided that Z is not attached to an aryl or a heteroaryl group); and
R² represents H or C₁₋₆ alkyl, which latter group is optionally substituted by one or more halo groups; or
when R² represents C₁₋₆ alkyl optionally substituted by halo, R¹ and R² may be linked together forming a further 5- to 7-membered ring, optionally containing 1 to 3 heteroatoms and/or 1 to 3 double bonds, which ring is itself optionally substituted by one or more substituents selected from G¹, Z (provided that the ring is not aromatic in nature) and B¹ (which B¹ group is optionally substituted as described above);
R³ represents C₁₋₆ alkyl, C₂₋₆ alkenyl, C₂₋₆ alkynyl, C₃₋₈ cycloalkyl, C₃₋₈ heterocycloalkyl, aryl or heteroaryl, all of which groups are optionally substituted by one or more substituents selected from G^{1a}, Z (provided that Z is not directly attached to an aryl or a heteroaryl group) and B¹ (which B¹ group is optionally substituted as described above);
X represents a direct bond, —O— or
Y represents —C(Q)-, —C(S)— or —S(O)₂—;
B¹, B² and B³ independently represent, on each occasion when used above, C₁₋₆ alkyl, C₂₋₆ alkenyl, C₂₋₆ alkynyl, C₃₋₈ cycloalkyl, C₃₋₈ heterocycloalkyl, aryl or heteroaryl;
G¹, G^{1a}, G² and G³ independently represent, on each occasion when used above, halo, cyano, —N₃, —NO₂, —ONO₂ or -A¹-R⁴;
wherein A¹ represents a spacer group selected from —C(Z)A²-, —N(R⁵)A³-, —OA⁴-, —S— or —S(O)ₙA⁵-, in which:
A² represents a single bond, —O—, —S— or —N(R⁵)—;
A³ represents A⁶, —C(Z)N(R⁵)C(Z)N(R⁵)—, —C(Z)N(R⁵)C(Z)O—, —C(Z)N(R⁵)S(O)ₙN(R⁵)—, —C(Z)S—, —S(O)ₙ—, —S(O)ₙN(R⁵)C(Z)N(R⁵)—, —S(O)ₙN(R⁵)C(Z)O—, —S(O)ₙN(R⁵)S(O)ₙN(R⁵)—, —C(Z)O—, —S(O)ₙN(R⁵)— or —S(O)ₙO—;
A⁴ represents A⁶, —S(O)ₙ—, —C(Z)O—, —S(O)ₙN(R⁵)— or —S(O)ₙO—
A⁵ represents a single bond, —N(R⁵)— or —O—;
A⁶ represents a single bond, —C(Z)- or —C(Z)N(R⁵)—;
Z represents, on each occasion when used above, a substituent connected by a double bond, which is selected from ═O, ═S, ═NR⁴, ═NN(R⁴)(R⁵), ═NOR⁴, ═NS(O)₂N(R⁴)(R⁵), ═NCN, ═CHNO₂ and ═C(R⁴)(R⁵);
R⁴ and R⁵ independently represent, on each occasion when used above, H or B⁴, which B⁴ group is itself optionally substituted by one or more substituents selected from G⁴, Q (provided that Q is not directly attached to an aryl or a heteroaryl group) and B⁵ (which B⁵ group is itself optionally substituted by one or more substituents selected from G⁵, Q (provided that Q is not directly attached to an aryl or a heteroaryl group) and B⁶); or when R⁴ and R⁵ both represent optionally substituted B⁴ groups, then any pair thereof may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more substituents selected from G⁶, Q (provided that the ring is not aromatic in nature) and B⁴ (which B⁴ group is optionally substituted as described above);
B⁴, B⁵ and B⁶ independently represent on each occasion when used above C₁₋₆ alkyl, C₂₋₆ alkenyl, C₂₋₆ alkynyl, C₃₋₈ cycloalkyl, C₃₋₈ heterocycloalkyl, aryl or heteroaryl;
G⁴, G⁵ and G⁶ independently represent on each occasion when used above, halo, cyano, N₃, —NO₂, —ONO₂ or -A⁷-R⁶;
wherein A⁷ represents a spacer group selected from —C(Q)A⁸-, —N(R⁷)A⁹-, —N(R^{7a})A^{9a}-, —OA¹⁰-, —S— or —S(O)ₙA¹¹-, in which:
A⁸ represents a single bond, —O—, —S— or —N(R⁷)—;
A⁹ represents A¹², —C(Q)S—, —S(O)ₙ—, —C(Q)O—, —S(O)ₙN(R⁷)— or —S(O)ₙO—;
A^{9a} represents —C(Q)N(R⁷)C(Q)N(R⁷)—, —C(Q)N(R⁷)C(Q)O—, —C(Q)N(R⁷)S(O)ₙN(R⁷)—, —S(O)ₙN(R⁷)C(Q)N(R⁷)—, —S(O)ₙN(R⁷)C(Q)O_, —S(O)ₙN(R⁷)S(O)ₙN(R⁷)—;
A¹⁰ represents A¹², —S(O)ₙ—, —C(Q)O—, —S(O)ₙN(R⁷)— or —S(O)ₙO—;
A¹¹ represents a single bond, —N(R⁷)— or —O—;
A¹² represents a single bond, —C(Q)- or —C(Q)N(R⁷)—;
Q represents, on each occasion when used above, a substituent connected by a double bond, which is selected from ═O, ═S, ═NR⁶, ═NN(R⁶)(R⁷), ═NOR⁶, ═NS(O)₂N(R⁶)(R⁷), ═NCN, ═CHNO₂ and ═C(R⁶)(R⁷);
R⁶, R⁷ and R^{7a} independently represent, on each occasion when used above, H, C₁₋₆ alkyl, C₂₋₆ alkenyl, C₂₋₆ alkynyl, C₃₋₈ cycloalkyl, C₃₋₈ heterocycloalkyl, aryl or heteroaryl, which latter seven groups are optionally substituted by one or more groups selected from halo, C₁₋₆ alkyl (optionally substituted by one or more halo groups), —N(R⁸)R⁹, —OR⁸, —ONO₂ and —SR⁸; or
provided that they do not represent H, any pair of R⁶ and R⁷ may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more groups selected from halo, C₁₋₆ alkyl (optionally substituted by one or more halo groups), —N(R⁸)R⁹, —OR⁸, —ONO₂ and —SR⁸;
R⁸ and R⁹ independently represent, on each occasion when used above, H or C₁₋₆ alkyl, which latter group is optionally substituted by one or more halo groups; and
n represents, on each occasion when used above, 1 or 2;
or a pharmaceutically-acceptable salt thereof,
provided that, when R² represents H, Y represents —C(Q)- and:
(A) X represents a direct bond and:
i) R³ represents phenyl, then R¹ does not represent phenyl, 2-methoxyphenyl, 2-thiazolyl or 6-methyl-2-pyridinyl;
ii) R³ represents 4-fluorophenyl, then R¹ does not represent 2-carbomethoxyphenyl, 3-carbomethoxyphenyl or 2,4-dimethylphenyl;
iii) R³ represents 2-chlorophenyl, then R¹ does not represent phenyl, 3-bromophenyl or 4-bromophenyl;
iv) R³ represents 3-chlorophenyl, then R¹ does not represent phenyl, 2-fluorophenyl, 2-chlorophenyl, 2,3-dichlorophenyl or 2,5-dichlorophenyl;
v) R³ represents 4-chlorophenyl, then R¹ does not represent 3-bromophenyl or 4-methoxyphenyl;
vi) R³ represents 3-iodophenyl, then R¹ does not represent 2-methoxyphenyl or 2,4-dimethylphenyl;
vii) R³ represents 2,4-dichlorophenyl, then R¹ does not represent 4-chlorophenyl or 2,3-dichlorophenyl;
viii) R³ represents 3,5-dinitrophenyl, then R¹ does not represent 2,3-dichlorophenyl;
ix) R³ represents 2,4-dimethyl-6-oxo-6H-pyran-3-yl, then R¹ does not represent 3-carbomethoxyphenyl;
x) R³ represents methyl, then R¹ does not represent 3,4-dichlorophenyl, 2-methoxyphenyl, 2-thiazolyl, 4-methyl-2-pyridinyl, 6-methyl-2-pyridinyl or 4-acetylphenyl;
xi) R³ represents ethyl, then R¹ does not represent phenyl, 2,3-dichlorophenyl, 4-methoxyphenyl, 2-carbomethoxyphenyl, 2-thiazolyl or 4-methyl-2-pyridinyl;
   (B) X represents —N(H)— and:
i) R³ represents phenyl, then R¹ does not represent 4-methoxyphenyl, 2,4-dimethylphenyl or 2-thiazolyl;
ii) R³ represents 3-chlorophenyl, then R¹ does not represent 4-methylphenyl;
iii) R³ represents 4-chlorophenyl, then R¹ does not represent 3-bromophenyl;
iv) R³ represents 3,4-dichlorophenyl, then R¹ does not represent 4-methyl-2-pyridinyl or 6-methyl-2-pyridinyl;
v) R³ represents 2′-sulfamoylbiphenyl-4-yl, then R¹ does not represent 5-bromo-2-pyridinyl;
vi) R³ represents 1-propyl, then R¹ does not represent phenyl;
vii) R³ represents 1-butyl, then R¹ does not represent 4-bromophenyl or 2,4-dimethylphenyl;
viii) R³ represents cyclohexyl, then R¹ does not represent 4-methoxyphenyl;
   (C) X represents —O— and:
i) R³ represents phenyl, then R¹ does not represent phenyl or 6-methyl-2-pyridinyl;
ii) R³ represents methyl, then R¹ does not represent phenyl, 2-fluorophenyl, 2,4-dimethylphenyl, 4-acetylphenyl or 2-thiazolyl;
iii) R³ represents ethyl, then R¹ does not represent phenyl, 2-fluorophenyl, 4-acetylphenyl or 4-methyl-2-pyridinyl;
iv) R³ represents 1-butyl, then R¹ does not represent 2-fluorophenyl, 2-methoxyphenyl, 4-methyl-2-pyridinyl or 6-methyl-2-pyridinyl;
v) R³ represents 2-butyl, then R¹ does not represent 2-thiazolyl or 4-acetylphenyl;
vi) R³ represents 2-methyl-1-propyl, then R¹ does not represent phenyl or 3-nitrophenyl,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of formula I may contain double bonds and may thus exist as E (entgegen) and Z (zusammen) geometric isomers about each individual double bond. All such isomers and mixtures thereof are included within the scope of the invention.

Compounds of formula I may also exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of three) of carbon atoms, be branched-chain. C₁₋₆-alkyl groups that may be mentioned include methyl, ethyl, n-propyl, isopropyl, n-butyl, sec-butyl, isobutyl, tert-butyl, n-pentyl, isopentyl, n-hexyl, and isohexyl.

C_{2-q} alkenyl groups (where q is the upper limit of the range) defined herein may be straight-chain or, when there is a sufficient number (i.e. a minimum of three) of carbon atoms, be branched-chain. Such alkenyl groups may contain one or more double bonds. C₂₋₆-alkenyl groups that may be mentioned include vinyl, 1-propenyl, 2-propenyl, propadienyl, 1-butenyl, 2-butenyl, 3-butenyl, 1,3-butadienyl, 1-pentenyl, 2-pentenyl, 4-pentenyl, and 5-hexenyl.

C_{2-q} alkynyl groups (where q is the upper limit of the range) defined herein may be straight-chain or, when there is a sufficient number (i.e. a minimum of four) of carbon atoms, be branched-chain. Such alkynyl groups may contain one or more triple bonds. C₂₋₆-alkynyl groups that may be mentioned include ethynyl, 1-propynyl, 2-propynyl, 1-butynyl, 2-butynyl, 3-butynyl, 1-pentynyl, 2-pentynyl, 4-pentynyl, 1-hexynyl, 3-hexynyl, and 5-hexynyl.

C_{3-q} cycloalkyl groups (where q is the upper limit of the range) that may be mentioned include monocyclic or bicyclic alkyl groups. Such cycloalkyl groups may be saturated or unsaturated containing one or more double or triple bond (forming for example a C_{3-q} cycloalkenyl or a C_{3-q} cycloalkynyl group). C₃₋₈-cycloalkyl groups that may be mentioned include cyclopropyl, cyclobutyl, cyclopentyl, cyclohexyl, cycloheptyl, cyclooctyl, cyclopentenyl, cyclohexenyl, cycloheptenyl, cyclooctenyl, cyclooctynyl, bicycloheptyl, bicyclooctyl, and bicyclooctenyl. Substituents may be attached at any point on the cycloalkyl group. Further in the case where the substituent is another cyclic compound, then the cyclic substituent may be attached through a single atom on the cycloalkyl group, forming a so-called “spiro”-compound.

C_{3-q} heterocycloalkyl groups (where q is the upper limit of the range) that may be mentioned include monocyclic or bicyclic alkyl groups in which at least one (e.g. one to four) of the atoms in the ring system is other than carbon (i.e. a hetereoatom). Further, such heterocycloalkyl groups may be saturated or unsaturated containing one or more double and/or triple bonds, forming for example a C_{3-q} heterocycloalkenyl or a C_{3-q} heterocycloalkynyl group. C_{3-q} heterocycloalkyl groups that may be mentioned include aziridinyl, azetidinyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridinyl, thietanyl, thiiranyl, thiolanyl, thiomorpholinyl, trithianyl (including 1,3,5-trithianyl), tropanyl and the like. Substituents on the heterocycloalkyl groups may, where appropriate, be located on any atom in the ring system including a heteroatom. Further in the case where the substituent is another cyclic compound, then the cyclic substituent may be attached through a single atom on the heteocycloalkyl group, forming a so-called “spiro”-compound. The point of attachment of a heterocycloalkyl group may be via any atom in the ring system including (where appropriate) a heteroatom, or an atom on any fused carbocyclic ring that may be present as part of the ring system. Heterocycloalkyl groups may also be in the N- or S-oxidised form.

The term “halo”, when used herein, includes fluoro, chloro, bromo and iodo.

Aryl groups that may be mentioned include C₆₋₁₀ aryl groups. Such groups may be monocyclic or bicyclic and have between 6 and 10 ring carbon atoms, in which at least one ring is aromatic. C₆₋₁₀ aryl groups include phenyl, naphthyl and the like, such as 1,2,3,4-tetrahydronaphthyl, indanyl, and indenyl. The point of attachment of aryl groups may be via any atom of the ring system.

Heteroaryl groups that may be mentioned include those which have between 5 and 10 members. Such groups may be monocyclic, bicyclic or tricyclic, in which at least one of the rings is aromatic and wherein at least one (e.g. one to four) of the atoms in the ring system is other than carbon (i.e. a heteroatom). Heterocyclic groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl (including 2,1,3-benzothiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothiophenyl, carbazolyl, chromanyl, cinnolinyl, furanyl, imidazolyl, imidazo[1,2-a]pyridinyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridinyl, pyrimidinyl, pyrrolyl, quinazolinyl, quinolinyl, quinolizinyl, quinoxalinyl, tetrahydroisoquinolinyl, tetrahydroquinolinyl, tetrazolyl, thiadiazolyl (including 1,2,3-s thiadiazolyl, 1,2,4-thiadiazolyl and 1,3,4-thiadiazolyl), thiazolyl, thiochromanyl, thiophenyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Heteroatoms that may be mentioned include oxygen, nitrogen, sulphur and selenium.

For the avoidance of doubt, in cases in which the identity of two or more substituents in a compound of formula I may be the same, the actual identities of the respective substituents are not in any way interdependent. For example, in the situation in which R¹ and R³ are both aryl groups substituted by one or more C₁₋₆ alkyl groups, the alkyl groups in question may be the same or different. Similarly, when groups are substituted by more than one substituent as defined herein, the identities of those individual substituents are not to be regarded as being interdependent.

Compounds of the invention that may be mentioned include those of formula I, and pharmaceutically-acceptable salts thereof, in which any one, or more (including all), of the specific compounds that are excluded by way of the above provisos are not so excluded.

Compounds that may be mentioned include those in which B⁴, R⁶ and/or R⁷ do not represent a heteroaryl group.

Preferred compounds of the invention include those in which:

R¹ represents an aryl or heteroaryl group, both of which are optionally substituted as hereinbefore defined;

G¹ represents halo, cyano or -A¹-R⁴;

G^{1a} represents halo, cyano, —NO₂ or -A¹-R⁴;

G² represents halo, cyano, —ONO₂ or -A¹-R⁴;

B² represents C₁₋₆ alkyl, C₂₋₆ alkenyl or C₂₋₆ alkynyl, all of which are optionally substituted by one or more G³ and/or B³ groups;

G³ represents halo, —ONO₂, —N(R⁵)(R⁴) or —OR⁴;

B³ represents C₁₋₆ alkyl, C₂₋₆ alkenyl or C₂₋₆ alkynyl;

when A¹ represents —N(R⁵)A³-, A³ represents A⁶, —C(Z)S—, —S(O)ₙ—, —C(Z)O— or —S(O)ₙN(R⁵)—;

when A¹ represents —OA⁴-, A⁴ represents A⁶;

when A¹ represents —S(O)ₙA⁵-, A⁵ represents a single bond or —N(R⁵)—;

Z represents ═O or ═NR⁴;

when any pair of R⁴ and R⁵ are linked together to form a ring, they are optionally substituted with G⁶ and/or B⁴;

G⁴ represents halo, cyano, —ONO₂ or -A⁷-R⁶;

B⁵ represents C₁₋₆ alkyl, C₂₋₆ alkenyl or C₂₋₆ alkynyl, all of which are optionally substituted by one or more G⁵ and/or B⁶ groups;

G⁵ represents halo, —ONO₂, —N(R⁷)(R⁶) or —R⁶;

B⁶ represents C₁₋₆ alkyl, C₂₋₆ alkenyl or C₂₋₆ alkynyl;

G⁶ represents halo, cyano or -A⁷-R⁶;

A⁷ represents-C(Q)A⁸-, —N(R⁷)A⁹-, —OA¹⁰-, —S— or —S(O)ₙA¹¹-;

when A⁷ represents —N(R⁷)A⁹-, A⁹ represents A¹², —C(Q)S—, —S(O)ₙ—, —C(Q)O— or —S(O)ₙN(R⁷)—;

when A⁷ represents —OA¹⁰-, A¹⁰ represents A¹²;

when A⁷ represents —S(O)ₙA¹¹-, A¹¹ represents a single bond or —N(R⁷)—;

Q represents ═O or ═NR⁶;

R⁶, R⁷ and R^{7a} independently represent H, C₁₋₆ allyl, C₂₋₆ alkenyl or C₂₋₆ alkynyl, all of which groups are optionally substituted by one or more groups selected from halo, C₁₋₆ alkyl, —N(R⁸)R⁹, OR⁸ and —ONO₂; and/or

when any pair of R⁶ and R⁷ are linked together to form a ring, that ring is optionally substituted by one or more groups selected from halo, C₁₋₆ alkyl (optionally substituted by one or more halo groups), —N(R⁸)R⁹, —OR⁸ and —ONO₂.

More preferred compound include those in which:

B¹ represents a C₁₋₆ alkyl, C₂₋₆ alkenyl, C₂₋₆ alkynyl, C₃₋₇ cycloalkyl, C₄₋₇ heterocycloalkyl, or phenyl, group, all of which are optionally substituted as described hereinbefore;

G² represents halo (e.g. fluoro or chloro); B² represents C₁₋₆ alkyl, C₂₋₆ alkenyl or C₂₋₆ alkynyl, all of which are optionally substituted by one or more halo groups;

when A¹ represents —C(Z)A²-, A² represents a single bond, —O— or —N(R⁵)—;

when A¹ represents —N(R⁵)A³-, A³ represents A⁶;

when A¹ represents —S(O)ₙA⁵, A⁵ represents a single bond;

A⁶ represents a single bond;

Z represents ═O;

B⁴ represents a C₁₋₆ alkyl, C₂₋₆ alkenyl, C₂₋₆ alkynyl, C₃₋₇ cycloalkyl, C₄₋₇ heterocycloalkyl, or phenyl, group, all of which are optionally substituted as described hereinbefore;

G⁴ represents halo;

B⁵ represents C₁₋₆ alkyl, C₂₋₆ alkenyl or C₂₋₆ alkynyl, all of which groups are optionally substituted by one or more halo groups;

when A⁷ represents —C(Q)A⁸-, A⁸ represents a single bond, —O— or —N(R⁷)—;

when A⁷ represents —N(R⁷)A⁹-, A⁹ represents A¹²;

when A⁷ represents —S(O)ₙA¹¹, A¹¹ represents a single bond;

A¹² represents a single bond;

Q represents ═O.

Further preferred compounds include those in which:

B⁴ represents C₂₋₆ alkenyl, C₂₋₆ alkynyl or, preferably, C₁₋₆ alkyl;

G⁶ represents halo;

R⁶, R⁷ and/or R^{7a} represent H, C₁₋₆ alkyl optionally substituted by one or more halo groups.

Further preferred compounds include those in which:

R⁴ and/or R⁵ independently represent H or C₁₋₆ alkyl, which latter group is optionally substituted by one or more fluoro groups.

Further preferred compounds include those in which:

B¹ represents a C₁₋₆ alkyl, C₂₋₆ alkenyl, C₃₋₆ cycloalkyl, C₅₋₆ heterocycloalkyl, or phenyl, group, all of which are optionally substituted as described hereinbefore;

G^{1a} represents halo, —NO₂ or -A¹-R⁴;

A¹ represents —C(Z)A²-, —N(R⁵)A³- and -OA⁴-;

when A¹ represents —C(Z)A²-, A² represents a single bond or —O—.

Preferred compounds of the invention include those in which R¹ represents an optionally substituted phenyl, naphthyl, pyrrolidinyl, piperidinyl, pyrrolyl, furanyl, thiophenyl, pyrazolyl, imidazolyl, oxazolyl, isoxazolyl, thiazolyl, pyridinyl (e.g. 2-pyridinyl, 3-pyridinyl or 4-pyridinyl), indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothiophenyl, pyridazinyl, pyrimidinyl, pyrazinyl, indazolyl, benzimidazolyl, quinazolinyl, quinoxalinyl, 1,3-benzodioxolyl, benzothiazolyl, and/or benzodioxanyl, group. It is preferred in the above list that R¹ does not represent naphthyl, pyrrolidinyl, piperidinyl, indazolyl, oxindolyl or benzothiazolyl. Particularly preferred values of R¹ include optionally substituted phenyl, pyridinyl (especially 2- and 3-pyridinyl), thiophenyl (especially 2-thiophenyl), pyrazolyl (especially 4-pyrazolyl), isoxazolyl (especially 5-isoxazolyl), benzodioxolyl (especially 1,3-benzodioxolyl), indazolyl, benzothiazolyl, and quinolinyl, groups.

Such R¹ groups are optionally substituted by one or more substituents selected from:

halo (e.g. fluoro, chloro or bromo);

cyano;

C₁₋₆ alkyl, which alkyl group may be linear or branched (e.g. C₁₋₄ alkyl (including methyl, ethyl, n-propyl, i-propyl, n-butyl, s-butyl, i-butyl or t-butyl), n-pentyl, i-pentyl, n-hexyl or i-hexyl), substituted by one or more fluoro group (e.g. —CH₂F, —CHF₂ or —CF₃), and/or substituted by a C₃₋₆ cycloalkyl (e.g. cyclopropyl) group, so forming, for example, a cyclopropylmethyl group;

C₂₋₆ alkenyl (e.g. 1-propenyl, 2-propenyl, 1-butenyl, 2-butenyl, 3-butenyl, 1-pentenyl, 2-pentenyl, 4-pentenyl or 5-hexenyl);

C₃₋₆ cycloalkyl (e.g. cyclopropyl, cyclobutyl, cyclopentyl or cyclohexyl);

phenyl;

a heterocyclic group selected from a pyrrolidinyl (including 1-pyrrolidinyl, 2-pyrrolidinyl and 3-pyrrolidinyl), a piperidinyl (including 1-piperidinyl, 2-piperidinyl, 3-piperidinyl, 4-piperidinyl and 1-methyl-4-piperidinyl), a piperazinyl (including 1-piperazinyl and 4-methyl-1-piperazinyl), a tetrahydrofuranyl (including 2-tetrahydrofuranyl and 3-tetrahydrofuranyl), a tetrahydropyranyl (including 1-tetrahydropyranyl, 2-tetrahydropyranyl and 3-tetrahydropyranyl), or a 4-morpholinyl, group;

thiomethyl, methylsulfinyl, methylsulfonyl;

—OR¹⁰;

—N(R¹⁰)R¹¹;

—C(O)OR¹⁰;

—C(O)R¹⁰;

—C(O)N(R¹⁰)R¹¹;

—S(O)₂N(R¹⁰)R¹¹; and/or

—N(R¹⁰)S(O)₂R¹²;

wherein R¹⁰ and R¹¹ independently represent, on each occasion when used above, H, phenyl, C₁₋₆ alkyl (such as methyl, ethyl, n-propyl, i-propyl, n-butyl or t-butyl), which alkyl group is optionally substituted by one or more fluoro atom, C₂₋₆ alkenyl or C₃₋₆ cycloalkyl; or

R¹⁰ and R¹¹ may be linked together to form, with the nitrogen atom to which they are attached, a 5- to 7-membered ring, optionally containing one additional heteroatom and optionally substituted with one or more C₁₋₆ alkyl groups, which alkyl groups are themselves optionally substituted by one or more halo (e.g. fluoro) groups (for example a morpholine, a piperazine, or a 4-methylpiperazine, group); and

R¹² represents phenyl, C₁₋₆ alkyl (such as methyl, ethyl, n-propyl, i-propyl, n-butyl or t-butyl), which alkyl group is optionally substituted by one or more fluoro atom, C₂₋₆ alkenyl or C₃₋₆ cycloalkyl.

When one or more of the above optional substituents on R¹ represents:

—OR¹⁰, then R¹⁰ is preferably H, methyl (optionally substituted by 1 to 3 fluoro atoms, e.g. CF₃), ethyl, n-propyl or i-propyl;

—N(R¹⁰)R¹¹, then R¹⁰ is preferably H, methyl, ethyl, n-propyl, i-propyl, n-butyl or t-butyl and/or R¹¹ is preferably H, methyl or ethyl; —C(O)OR¹⁰, then R¹⁰ is preferably methyl, ethyl or i-propyl; —C(O)R¹⁰, then R¹⁰ is preferably methyl, ethyl or t-butyl;

—C(O)N(R¹⁰)R¹¹ or —S(O)₂N(R¹⁰)R¹¹, then R¹⁰ and R¹¹ are preferably H or methyl;

—N(R¹⁰)S(O)₂R², then R¹⁰ is preferably H or methyl and R¹² is preferably methyl.

Particularly preferred optional substituents on R¹ include carbomethoxy, methyl, dimethylamino, cyano, chloro, fluoro, trifluoromethyl, bromo, methoxy and trifluoromethoxy.

Preferred compounds of the invention include those in which R³ represents an optionally substituted C₁₋₆ alkyl, C₃₋₆ cycloalkyl, phenyl, naphthyl, pyrrolidinyl, piperidinyl (especially 4-piperidinyl), piperazinyl (especially 4-piperazinyl), pyrrolyl, furanyl, thiophenyl, pyrazolyl, imidazolyl, oxazolyl, isoxazolyl, thiazolyl, pyridinyl (e.g. 2-pyridinyl, 3-pyridinyl or 4-pyridinyl), indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothiophenyl, pyridazinyl, pyrimidinyl, pyrazinyl, indazolyl, benzimidazolyl, quinazolinyl, quinoxalinyl, 1,3-benzodioxolyl, benzothiazolyl and/or benzodioxanyl group. Particularly preferred groups include an optionally substituted C₁₋₆ alkyl (such as methyl, pentyl or hexyl), cyclohexyl, phenyl, thiophenyl (especially 2-thiophenyl), furanyl (especially 2-furanyl and 3furanyl), pyrrolyl (especially 2-pyrrolyl), naphthyl (especially 1-naphthyl), benzofuranyl, piperazinyl (especially 4-piperazinyl), piperidinyl (especially 4-piperidinyl), or benzodioxolyl (especially 1,3-benzodioxolyl), group.

Such R³ groups are optionally substituted by one or more substituents selected from:

halo;

—NO₂;

cyano;

C₁₋₆ alkyl, which alkyl group may be linear or branched (e.g. C₁₋₄ alkyl (including methyl, ethyl, n-propyl, i-propyl, n-butyl, s-butyl, i-butyl or t-butyl), n-pentyl, i-pentyl, n-hexyl or i-hexyl), substituted by one or more halo (e.g. fluoro) group (e.g. —CH₂F, —CHF₂ or —CF₃), C₁₋₆ alkyl group (e.g. methyl or ethyl), C₂₋₆ alkenyl group or C₃₋₆ cycloalkyl group (e.g. cyclopropylmethyl, so forming, for example, a cyclopropylmethyl group), which latter three groups are themselves optionally substituted with one or more halo (e.g. fluoro) and/or C₁₋₆ alkyl groups;

C₂₋₆ alkenyl (e.g. 1-propenyl, 2-propenyl, 1-butenyl, 2-butenyl, 3-butenyl, 1-pentenyl, 2-pentenyl, 4-pentenyl or 5-hexenyl), optionally substituted with one or more C₁₋₆ alkyl groups;

C₃₋₆ cycloalkyl (e.g. cyclopropyl, cyclobutyl, cyclopentyl or cyclohexyl), optionally substituted with one or more halo (e.g. fluoro) group; phenyl, optionally substituted with one or more halo (e.g. fluoro or, especially, chloro) groups;

a heterocyclic group selected from a pyrrolidinyl (including 1-pyrrolidinyl, 2-pyrrolidinyl and 3-pyrrolidinyl), a piperidinyl (including 1-piperidinyl, 2-piperidinyl, 3-piperidinyl, 4-piperidinyl and 1-methyl-4-piperidinyl), a piperazinyl (including 1-piperazinyl and 4-methyl-1-piperazinyl), a tetrahydrofuranyl (including 2-tetrahydrofuranyl and 3-tetrahydrofuranyl), a tetrahydropyranyl (including 1-tetrahydropyranyl, 2-tetrahydropyranyl and 3-tetrahydropyranyl), or a 4-morpholinyl, group;

thiomethyl, methylsulfinyl, methylsulfonyl;

═O;

—OR¹³;

—N(R¹³)R¹⁴;

—C(O)OR¹³;

—C(O)R¹³;

—C(O)N(R¹³)R¹⁴;

—S(O)₂N(R¹³)R¹⁴; and/or —N(R¹³)S(O)₂R¹⁵;

wherein R¹³ and R¹⁴ independently represent, on each occasion when used above, H, phenyl, C₁₋₆ alkyl (such as methyl, ethyl, n-propyl, i-propyl, n-butyl or t-butyl) which alkyl group is optionally substituted by one or more fluoro atom, C₂₋₆ alkenyl or C₃₋₆ cycloalkyl; or

R¹³ and R¹⁴ may be linked together to form, with the nitrogen atom to which they are attached, a 5- to 7-membered ring, optionally containing one additional heteroatom and optionally substituted with one or more C₁₋₆ alkyl groups, which alkyl groups are themselves optionally substituted by one or more halo (e.g. fluoro) groups (for example a morpholine, a piperazine, or a 4-methylpiperazine, group); and

R¹⁵ represents phenyl, C₁₋₆ alkyl (such as methyl, ethyl, n-propyl, i-propyl, n-butyl or t-butyl), which alkyl group is optionally substituted by one or more fluoro atom, C₂₋₆ alkenyl or C₃₋₆ cycloalkyl.

When one or more of the above optional substituents on R³ represents: —OR¹³, then R¹³ is preferably H, methyl (optionally substituted by 1 to 3 fluoro atoms, e.g. —CF₃), ethyl, n-propyl or i-propyl;

—N(R¹³)R⁴, then R¹³ is preferably H, methyl, ethyl, n-propyl, i-propyl, n-butyl or t-butyl and/or R¹⁴ is preferably H, methyl or ethyl; —C(O)OR¹³, then R¹³ is preferably methyl, ethyl or i-propyl;

—C(O)R¹³, then R¹³ is preferably methyl, —CF₃, ethyl or t-butyl;

—C(O)N(R³)R¹⁴ or —S(O)₂N(R³)R⁴, then R and R¹⁴ are preferably H or methyl;

—N(R¹³)S(O)₂R¹⁵, then R¹³ is preferably H or methyl and R¹⁵ is preferably methyl.

When R³ represents C₁₋₆ alkyl or C₂₋₆ alkenyl substituted by other alkyl or alkenyl groups (which may be further substituted by alkyl/alkenyl, as indicated above), groups that may be mentioned include 1-octyl, 1-tridecanyl, 1-pentadecanyl, 1-heptadecanyl, 1-heptadec-8-enyl, 1-heptadeca-8,11-dienyl, 1-heptadeca-8,11,14-trienyl and 1-nonadeca-4,7,10,13-tetraenyl groups.

Particularly preferred optional substituents on R³ include methyl, ethyl, ethoxy, trifluoromethyl, fluoro, chloro, iodo, phenyl, chlorophenyl (such as 2-chlorophenyl and 4-chlorophenyl), n-pentyl, i-propyl, nitro, t-butyl, —CH₂CH═CHC₈H₁₇, trifluoroacetyl, carbomethoxy, carboethoxy and trifluoromethoxy.

Further preferred compounds of the invention include those in which:

R² represents H or C₁₋₃ alkyl;

X represents a direct bond, —O—, —N(H)— or —N(Me)—.

More preferred values of R² include H, methyl and ethyl, particularly H or methyl.

Compounds of the invention that may also be mentioned include those in which:

R¹ is phenyl, 2-chlorophenyl, 2-chloro-4-fluorophenyl, 3-chloro-4-fluorophenyl, 2,6-dichlorophenyl, 5-chloro-2-cyanophenyl, 2-fluoro-5-trifluoromethylphenyl, 2-bromo-4-trifluoromethoxyphenyl, 2-methoxy-6-methylphenyl, 3-cyanophenyl, 4-trifluoromethylphenyl, 4-dimethylaminophenyl, 4-carbomethoxyphenyl, 1,3,5-trimethyl-1H-pyrazol-4-yl, 3-methylisoxazol-5-yl, 3-pyridinyl, 2-chloro-3-pyridinyl, 3-methyl-2-pyridinyl, 3-carbomethoxythiophen-2-yl or 1,3-benzodioxolyl;

R² is hydrogen or methyl;

R³ is methyl, n-butyl, n-pentyl, 1-octyl, oleoyl, (1R,2S,5R)-(−)-menthyl, 2-chlorobenzyl, benzyl, phenyl, 3-fluorophenyl, 3-chlorophenyl, 4-chlorophenyl, 2-fluoro-5-iodophenyl, 5-fluoro-2-methylphenyl, 4-tert-butyl-phenyl, 4-pentylphenyl, 3-trifluoromethylphenyl, 4-trifluoromethoxyphenyl, 4-nitrophenyl, 2-ethoxyphenyl, 1-naphthyl, 2-furanyl, 2,5-dimethyl-3-furanyl, 2-carbomethoxy-5-furanyl, 1-methyl-1H-pyrrol-2-yl, 3-methyl-2-benzofuranyl, 3-methyl-2-thiophenyl, 1(N)-methyl-4-piperazinyl, 1(N)-(2,2,2-trifluoroacetyl)piperidin-4-yl, ethylhexanoate or 1,3-benzodioxolyl;

Y is —C(O)—, —C(S)— or —S(O)₂—; and

X is a bond, —N(H)—, —N(Me)—, or —O—.

Further compounds of the invention that may also be mentioned include those in which:

R¹ is 2-chlorophenyl, 5-chloro-2-cyanophenyl, 4-dimethylaminophenyl, 4-carbomethoxyphenyl, 1,3,5-trimethyl-1H-pyrazol-4-yl, 3-methylisoxazol-5-yl, 3-pyridinyl, or 3-carbomethoxythien-2-yl;

R² is hydrogen or methyl;

R³ is methyl, 1-octyl, oleoyl, (1R,2S,5R)-(−)-menthyl, 2-chlorobenzyl, phenyl, 3-fluorophenyl, 3-chlorophenyl, 4-chlorophenyl, 2-fluoro-5-iodophenyl, 5-fluoro-2-methylphenyl, 4-tert-butylphenyl, 4-pentylphenyl, 3-trifluoromethylphenyl, 4-nitrophenyl, 2-ethoxyphenyl, 1-naphthyl, 2-furanyl, 2,5-dimethyl-3-furanyl, 2-carbomethoxy-5-furanyl, 1-methyl-1H-pyrrol-2-yl, 3-methyl-2-benzofuranyl, or 3-methyl-2-thiophenyl;

Y is —C(Q)-, —C(S)— or —S(O)₂—; and

X is a bond, —N(H)—, —N(Me)—, or —O—.

Particularly preferred compounds of the invention include those of the examples described hereinafter.

Compounds of formula I may be made in accordance with techniques that are well known to those skilled in the art, for example as described hereinafter.

According to a further aspect of the invention there is provided a process for the preparation of a compound of formula I, which process comprises:
(i) for compounds of formula I in which, when Y is —S(O)₂—, X represents a direct bond or —N(R⁴)—, in which R⁴ represents B⁴, reaction of a compound of formula II, 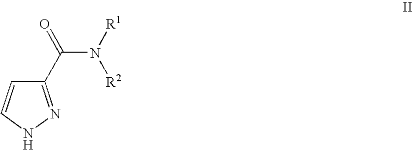
wherein R¹ and R² are as hereinbefore defined, with a compound of formula III,
R³—X^{a}—Y-L¹ III
wherein X^{a} represents a direct bond or —N(B⁴)— when Y represents —S(O)₂— or, for all other values of Y, represents X as hereinbefore defined, R³ and Y are as hereinbefore defined and L¹ represents a suitable leaving group, such as halo (e.g. chloro or bromo), or, when X^{a} is a direct bond, a carboxylate (e.g. a —O—C(O)—R³) group or a sulfonylate (e.g. a —O—S(O)₂—R³) group, or, when X^{a} is —N(B⁴)—, an N-imidazolyl group, for example at around room temperature or above (e.g. up to 40-180° C.) in the presence of a suitable base (e.g. sodium hydride, sodium bicarbonate, potassium carbonate, pyrrolidinopyridine, pyridine, triethylamine, tributylamine, trimethylamine, dimethylaminopyridine, di-iso-propylamine, 1,8-diazabicyclo[5.4.0]undec-7-ene, sodium hydroxide, N-ethyl-di-iso-propylamine, N-(methylpolystyrene)-4-(methylamino)pyridine or mixtures thereof) and an appropriate solvent (e.g. tetrahydrofuran, pyridine, toluene, dichloromethane, chloroform, dimethylformamide, trifluoromethylbenzene or triethylamine). Preferred base/solvent systems for compounds of formula III in which Y is —C(O)— and X is a direct bond include sodium hydride in tetrahydrofuran, DMF or mixtures thereof. Preferred base/solvent systems for compounds of formula III in which Y is —C(O)— and X^{a} is —O— or —NR⁴— or when Y is —S(O)₂— and X^{a} is a direct bond include dimethylaminopyridine/dichloromethane, or a mixture of triethylamine and dimethylaminopyridine in dichloromethane;
(ii) for compounds of formula I in which X represents a single bond and Y represents —(O)—, reaction of a compound of formula II as hereinbefore defined with a compound of formula IV,
R³C(O)OH IV
wherein R³ is as hereinbefore defined for example under similar conditions to those described under process step (i) above, in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytris-pyrrolidinophosphonium hexafluorophosphate, bromo-tris-pyrrolidinophosponium hexafluorophosphate, 2-(1H-benzotriazol-1-yl)-1,1,3,3-tetramethyluronium tetrafluorocarbonate) or 1-cyclohexylcarbodiimide-3-propyloxymethyl polystyrene, a suitable base (e.g. as mentioned in process step (i) above) and an appropriate solvent (e.g. as mentioned in process step (i) above). Alternatively an azodicarboxylate may be employed under Mitsunobo conditions known to those skilled in the art;
(iii) for compounds of formula I in which X represents a direct bond and Y represents a —C(O)— or a —C(S)— group, reaction of a compound of formula II as hereinbefore defined with a compound of formula V,
R³═Y^{a} V
wherein Y^{a} represents either —(O)— (so forming a ketene) or —C(S)— (so forming a thioketene) and R³ is as hereinbefore defined, under conditions known to those skilled in the art;
(iv) for compounds of formula I, in which X represents —NH— and Y represents —(O)— or —C(S)—, reaction of a compound of formula II as hereinbefore defined with a compound of formula VI,
R³N═Y^{a} VI
wherein R³ and Y^{a} are as hereinbefore defined (so forming an isocyanate or an isothiocyanate, as appropriate), under conditions known to those skilled in the art. For example, for compounds of formula VI in which Y is —C(O)—, reaction may be performed in a suitable solvent (e.g. toluene) at elevated temperature (e.g. 100° C.). For compounds of formula VI in which Y is —C(S)—, reaction may be performed in a suitable solvent (e.g. acetone) in the presence of a suitable base (e.g. potassium carbonate) at room temperature;
(v) for compounds of formula I in which Y represents —C(O)— or —C(S)—, reaction of a compound of formula II with:
(a) a compound of formula VII,
Cl—Y^{a}—Cl VII
(b) a compound of formula VIII, 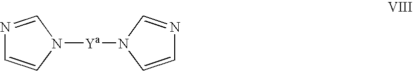
wherein, in both cases, Y^{a} is as hereinbefore defined; or
(c) when Y represents —C(O)—, triphosgene,
followed by:
(1) for compounds of formula I in which X represents a direct bond, reaction with a organometallic reagent of formula IX,
R³M IX
wherein M represents a metal such as Mn, Fe, Ni, Cu, Zn, Pd or Ce, or a salt or complex thereof and R³ is as hereinbefore defined;
(2) for compounds of formula I wherein X represents O, reaction with an alcohol of formula X,
R³OH X
wherein R³ is as hereinbefore defined; or
(3) for compounds of formula I wherein X represents —N(R⁴)—, reaction with an amine of formula XI,
R³N(H)R⁴ XI
wherein R³ and R⁴ are as hereinbefore defined, in all cases under reaction condition that are known to those skilled in the art;
(vi) for compounds of formula I in which X represents —N(R⁴)—, and R⁴ is other than hydrogen, reaction of a corresponding compound of formula I in which X represents —N(H)— with a compound of formula XII,
R⁴-L¹ XII
wherein R⁴ and L¹ are as hereinbefore defined under standard reaction conditions;
(vii) for compounds of formula I in which Y represents C(S)—, reaction of a corresponding compound of formula I in which Y represents —C(O)— with a suitable reagent for the conversion of a carbonyl group to a thiocarbonyl group, such as P₂S₅ or Lawesson's reagent, under conditions known to the person skilled in the art;
(viii) reaction of a compound of formula XIII, 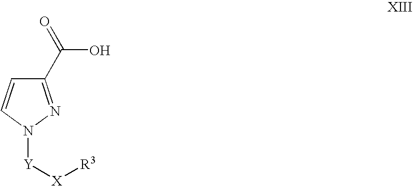
wherein R³, Y and X are as hereinbefore defined, with a compound of formula XIV,
HN(R¹)(R²) XIV
wherein R¹ and R² are as hereinbefore defined under coupling conditions, for example as described in process step (ii) above. Alternatively, compounds of formula XIII may first be activated by treatment with a suitable reagent (e.g. oxalyl chloride, thionyl chloride, etc) in an appropriate solvent (e.g. dichloromethane, dimethylformamide, THF or benzene), resulting in the formation of the respective acyl chloride. This activated intermediate may then be reacted with a compound of formula XIV under standard conditions, such as those described hereinbefore in process step (i) above; or
(ix) reaction of a compound of formula XV, 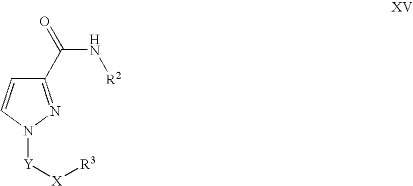
wherein R², R³, Y and X are as hereinbefore defined, with a compound of formula XVI,
R¹-L² XVI
wherein L² represents a suitable leaving group, such as halo (e.g. chloro, bromo and iodo), —OSO₂CF₃, —B(OH)₂, —Sn(R^{z})₃ (wherein R^{z} is preferably methyl or butyl) or —Bi(R¹)₂, and R¹ is as hereinbefore defined, for example in the presence of a catalyst containing, preferably, Pd or Cu, and a base.

Compounds of formula II and protected derivatives thereof may be prepared by reaction of a compound of formula XIV as hereinbefore defined with either:

(I) dipyrazolo[1,5-a;1′,5′-d]pyrazine-4,9-dione, for example under standard conditions, for example as described hereinbefore in process step (i); or

(II) 1H-pyrazole-3-carboxylic acid, an N-protected, and/or an O-activated derivative, thereof, for example under conditions such as those described hereinbefore in process step (viii) above.

Compounds of formula XIII may be prepared by reaction of 1H-pyrazole-3-carboxylic acid, or an O-protected derivative thereof, with an appropriate reagent under similar conditions to those described in respect any of process steps (i) to (vii) above.

Compounds of formula XV may be prepared by reaction of a compound of formula XVII,
H₂NR² XVII
wherein R² is as hereinbefore defined with a compound of formula XIII as hereinbefore defined, for example under conditions such as those described hereinbefore in respect of process step (viii) above.

Dipyrazolo[1,5-a;1′,5′-d]pyrazine-4,9-dione may be prepared from 1H-pyrazole-3-carboxylic acid under dimerising conditions, for example in the presence of thionyl chloride (optionally in the presence of DMF) at reflux. Other dimerising reagents include carbodiimides, such as 1,3-dicyclohexylcarbodiimide.

1H-Pyrazole-3-carboxylic acid and protected derivatives thereof may be prepared from 3-methyl-1(2)H-pyrazole, for example as described hereinafter.

Compounds of formulae III, IV, V, VI, VII, VIII, IX, X, XI, XII, XIV, XVI and XVII are either commercially available, are known in the literature, or may be obtained either by analogy with the processes described herein, or by conventional synthetic procedures, in accordance with standard techniques, from available starting materials using appropriate reagents and reaction conditions.

The substituents R¹, R² and R³ as hereinbefore defined may be modified one or more times, after or during the processes described above for preparation of compounds of formula I by way of methods that are well known to those skilled in the art. Examples of such methods include substitutions, reductions, oxidations, alkylations, hydrolyses, esterifications, and etherifications. The precursor groups can be changed to a different such group, or to the groups defined in formula I, at any time during the reaction sequence.

Compounds of the formula I may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Medical and Pharmaceutical Uses

Compounds of formula I and salts thereof are useful because they possess pharmacological activity. Such compounds are therefore indicated as pharmaceuticals.

To the applicant's knowledge, compounds of formula I have not been disclosed before for use as pharmaceuticals. According to a further aspect of the invention there is provided a compound of the invention, as hereinbefore defined but without the provisos, for use as a pharmaceutical.

Although compounds of formula I and salts thereof may possess pharmacological activity as such, certain pharmaceutically-acceptable (e.g. “protected”) derivatives of compounds of formula I may exist or be prepared which may not possess such activity, but may be administered parenterally or orally and thereafter be metabolised in the body to form compounds of formula I. Such compounds (which may possess some pharmacological activity, provided that such activity is appreciably lower than that of the “active” compounds to which they are metabolised), may therefore be described as “prodrugs” of compounds of formula I. All prodrugs of compounds of formula I are included within the scope of the invention.

By “prodrug of a compound of formula I”, we include compounds that form a compound of formula I, in an experimentally-detectable amount, within a predetermined time (e.g. about 1 hour), following oral or parenteral administration.

Compounds of formula I and salts thereof are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of formula I may thus be useful in the treatment of those conditions in which inhibition of a lipoxygenase, and particularly 15-lipoxygenase, is required.

Compounds of formula I, and pharmaceutically acceptable salts thereof, are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of formula I may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of formula I and pharmaceutically acceptable salts thereof may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of formula I are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of formula I will normally be administered orally, intravenously, subcutaneously, buccally, rectally, dermally, nasally, tracheally, bronchially, by any other parenteral route or via inhalation, in a pharmaceutically acceptable dosage form.

Compounds of formula I may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without the provisos, in admixture with a pharmaceutically acceptable adjuvant, diluent or carrier.

Compounds of formula I may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of the invention, as hereinbefore defined but without the provisos; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of formula I or salt thereof in conjunction with the other therapeutic agent, and may thus be presented either as separate formulations, wherein at least one of those formulations comprises compound of formula I/salt, and at least one comprises the other therapeutic agent, or may be presented (i.e. formulated) as a combined preparation (i.e. presented as a single formulation including compound of formula I/salt and the other therapeutic agent).

Thus, there is further provided:

(1) a pharmaceutical formulation including a compound of the invention, as hereinbefore defined but without the provisos, another therapeutic agent that is useful in the treatment of inflammation, and a pharmaceutically-acceptable adjuvant, diluent or carrier; and

(2) a kit of parts comprising components:

(a) a pharmaceutical formulation including a compound of the invention, as hereinbefore defined but without the provisos, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of formula I and salts thereof may be administered at varying doses. Oral dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For oral administration, the compositions typically contain between about 0.01 mg to about 500 mg, and preferably between about 1 mg to about 100 mg, of the active ingredient. Intravenously, the most preferred doses will range from about 0.1 to about 10 mg/kg/minute during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of formula I and salts thereof may have the advantage that they are effective and/or selective inhibitors of lipoxygenases, and particularly 15-lipoxygenase.

Compounds of formula I and salts thereof may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

Biological Test

The assay employed takes advantage of the ability of lipid hydroperoxides to oxidize the non-fluorescent diphenyl-1-pyrenylphosphine (DPPP) to its corresponding fluorescent phosphine oxide. Fluorescence is measured using a dual-scanning microplate spectrofluorometer, Spectramax Gemini, from Molecular Devices. DPPP was purchased from Molecular Probes. Linoleic acid was from Biomol and PBS (phosphate buffered saline) from Gibco Life Technologies. The assay is performed in 96-well plates at room temperature (20-22° C.). The following are added (in the following order) to each well:

a) 35 μl of Dulbecco's phosphate buffered saline (PBS);

b) inhibitor (i.e. compound) or vehicle (0.5 μl DMSO);

c) 10 μL of a 5× concentrated 15-lipoxygenase solution in PBS. The plates are incubated for 5 minutes at room temperature;

d) 5 μl of 2 mM linoleic acid in PBS. The plate is then incubated for 20 minutes at room temperature;

e) the enzymatic reaction is terminated by the addition of 50 μl methanol; and

f) 50 μl of 200 μM DPPP in methanol is added to each well.

After 30 minutes at room temperature, the fluorescence can be read using an excitation wavelength of 358 nm and an emission wavelength of 379 nm.

### EXAMPLES

The invention is illustrated by way of the following examples, in which the following abbreviations may be employed:

DMF dimethylformamide

DMSO dimethylsulfoxide

EtOAc ethyl acetate

MS mass spectrum

NMR nuclear magnetic resonance

THF tetrahydrofuran

Starting materials and chemical reagents specified in the syntheses described below are commercially available from, e.g. Sigma-Aldrich Fine Chemicals.

### Example 1

### 1-Benzoyl-1H-pyrazole-3-carboxylic acid pyridin-3-ylamide

### (a) 1H-Pyrazole-3-carboxylic acid

An aqueous solution of KMnO₄ (40.9 g, 0.26 mol) was added to a stirred is solution of 3-methyl-1(2)H-pyrazole (9.8 ml, 0.12 mol) in 0.5 L water. The mixture was heated at reflux for 5 h. The black suspension was cooled, filtered and the filtrate concentrated to a small volume. The solution was acidified with 3 N HCl and the white solid that formed was collected and washed with Et₂O to give the sub-title compound in 100% yield.

¹H NMR (DMSO-d₆, 200 MHz) δ 7.75 (d, J=1.5 Hz, 1H) and 6.75 (d, J=1.5 Hz, 1H).

### (b) 1H-Pyrazole-3-carbonyl chloride

Thionyl chloride (20 mL) was added to a solution of 1H-pyrazole-3-carboxylic acid (5.0 g, 44.6 mmol; see step (a) above) in THF (40 mL) at room temperature. The mixture was heated at reflux for 1 h, and concentrated to dryness to afford the sub-title compound. The solid was used without further purification.

### (c) 1H-Pyrazole-3-carboxylic acid pyridin-2-ylamide

NaH (60% dispersion in mineral oil, 1.34 g, 33.5 mmol) was added to a solution of pyridin-2-ylamine (3.0 g, 31.9 mmol) in THF (150 mL). The solution was stirred at room temperature for 1 hour and freshly prepared pyrazole-3-carbonyl chloride (4.66 g, 35.7 mmol; see step (b) above) was added. The mixture was stirred for 1 h and 10 mL water was added. The mixture was concentrated to a small volume and extracted twice with EtOAc. The combined extracts were washed with H₂O, dried with Na₂SO₄ and concentrated to dryness to afford the sub-title compound.

¹H NMR (CDCl₃, 200 MHz) δ 10.07 (s, 1H), 8.49 (d, J=8 Hz, 1H), 8.38 (d, J=6 Hz, 1H), 7.83 (dd, J=6 Hz, J=8 Hz, 1H), 7.68 (d, J=2.5 Hz, 1H), 7.14 (dd, J=6 Hz, J=8 Hz, 1H), 7.02 (d, J=2.5 Hz, 1H)

### (d) 1-Benzoyl-1H-pyrazole-3-carboxylic acid pyridin-3-ylamide

A solution of 1H-pyrazole-3-carboxylic acid pyridin-2-ylamide (100 mg, 0.53 mmol; see step (c) above) in anhydrous THF (2 mL) was added to NaH (60% dispersion in mineral oil, 22 mg, 0.56 mmol) with stirring at room temperature. After 1 h, benzoyl chloride (65 μL, 0.56 mmol) was added and the mixture was stirred for an additional hour. Water (1 mL) was added and the mixture was concentrated to dryness. EtOAc (2 mL) and Na₂CO₃ (0.2 M, 1 mL) were added and the phases were separated. The organic layer was washed with NaCl (aq. sat.), passed through a short column of silica gel and concentrated to dryness. The residue was triturated with Et₂O to afford the title compound (40 mg) as a white solid.

MS (M⁺+H) m/z 293.

### Example 2

### 1-(2,5-Dimethyl-3-furoyl)-1H-pyrazole-3-carboxylic acid pyridin-3-ylamide

The title compound was prepared as described in Example 1(d) from 1H-pyrazole-3-carboxylic acid pyridin-2-ylamide (see Example 1(c)) and 2,5-dimethyl-3-furoyl chloride.

MS (M⁺+H) m/z 311.

### Example 3

### 1-(1-Methyl-1H-pyrrol-2-yl)-1H-pyrazole-3-carboxylic acid pyridin-3-ylamide

The title compound was prepared as described in Example 1(d) from 1H-pyrazole-3-carboxylic acid pyridin-2-ylamide (see Example 1(c)) and 1-methyl-2-pyrroyl chloride.

MS (M⁺+H) m/z 296.

### Example 4

### 1-(3-Methyl-2-thienoyl)-1H-pyrazole-3-carboxylic acid pyridin-3-ylamide

The title compound was prepared as described in Example 1(d) from 1H-pyrazole-3-carboxylic acid pyridin-2-ylamide (see Example 1(c)) and 3-methyl-2-thienoyl chloride.

MS (M⁺+H) m/z 313.

### Example 5

### 1-(4-Pentylbenzoyl)-1H-pyrazole-3-carboxylic acid pyridin-3-ylamide

The title compound was prepared as described in Example 1(d) from 1H-pyrazole-3-carboxylic acid pyridin-2-ylamide (see Example 1(c)) and 4-pentylbenzoyl chloride.

MS (M⁺+H) m/z 363.

### Example 6

### 2-{[1-(2-Ethoxybenzoyl)-1H-pyrazole-3-carbonyl]amino}thiophene-3-carboxylic acid methyl ester

### (a) Dipyrazolo[1,5-a;1′,5′-d]pyrazine-4,9-dione

A mixture of pyrazole-3-carboxylic acid (44.0 g, 0.39 mol; see Example 1(a) above), DMF (0.5 mL), and thionyl chloride (150 mL) was heated at reflux for 3 days. The solvents were evaporated and the residue dried under high-vacuum, to give the sub-title compound which was used without further purification.

### (b) 2-[(1H-Pyrazole-3-carbonyl)amino]thiophene-3-carboxylic acid methyl ester

NaH (60% dispersion in mineral oil, 0.61 g, 15.4 mmol) was added to a solution of dipyrazolo[1,5-a;1′,5′-d]pyrazine-4,9-dione (2.4 g, 12.7 mmol; see step (a) above) and 2-aminothiophene-3-carboxylic acid methyl ester (2.0 g, 12.7 mmol) in THF (100 mL) at room temperature. The mixture was stirred for 1 h and 10 mL water was added. The mixture was concentrated to a small volume and extracted twice with EtOAc. The combined extracts were washed with H₂O, dried with Na₂SO₄ and concentrated to dryness providing the sub-title compound.

¹H NMR (DMSO-d₆, 200 MHz) δ 8.0 (d, J=2 Hz, 1H), 7.21 (d, J=5.8 Hz, 1H), 7.06 (d, J=5.8 Hz, 1H), 6.87 (d, J=2 Hz, 1H) and 3.86 (s, 3H).

### (c) 2-{[1-(2-Ethoxybenzoyl)-1H-pyrazole-3-carbonyl]amino}thiophene-3-carboxylic acid methyl ester

The title compound was prepared as described in Example 1(d) from 2-[(1H-pyrazole-3-carbonyl)amino]thiophene-3-carboxylic acid methyl ester (see step (b) above) and 2-ethoxybenzoyl chloride.

MS (M⁺+H) m/z 400.

### Example 7

### 2-{[1-(3-Trifluoromethylbenzoyl)-1H-pyrazole-3-carbonyl]amino}-thiophene-3-carboxylic acid methyl ester

The title compound was prepared as described in Example 1(d) from 2-[(1H-pyrazole-3-carbonyl)amino]thiophene-3-carboxylic acid methyl ester (see Example 6(b)) and 3-trifluorobenzoyl chloride.

MS (M⁺+H) m/z 424.

### Example 8

### 2-{[1-(5-Fluoro-2-methylbenzoyl)-1H-pyrazole-3-carbonyl]amino}-thiophene-3-carboxylic acid methyl ester

The title compound was prepared as described in Example 1(d) from 2-[(1H-pyrazole-3-carbonyl)amino]thiophene-3-carboxylic acid methyl ester (see Example 6(b)) and 5-fluoro-2-methylbenzoyl chloride.

MS (M⁺+H) m/z 388.

### Example 9

### 2-{[1-(3-Methyl-2-thienoyl)-1H-pyrazole-3-carbonyl]amino}thiophene-3-carboxylic acid methyl ester

The title compound was prepared as described in Example 1(d) from 2-[(1H-pyrazole-3-carbonyl)amino]thiophene-3-carboxylic acid methyl ester (see Example 6(b)) and 3-methyl-2-thienoyl chloride.

MS (M⁺+H) m/z 376.

### Example 10

### 1-(2,5-Dimethyl-3-furoyl)-1H-pyrazole-3-carboxylic acid (1,3,5-trimethyl-1H-pyrazol-4-yl)amide

### (a) 1H-Pyrazole-3-carboxylic acid (1,3,5-trimethyl-1H-pyrazol-4-yl)amide

The title compound was prepared as described in Example 6(b) from dipyrazolo[1,5-a;1′,5′-d]pyrazine-4,9-dione (see Example 6(a)) and 1,3,5-trimethyl-1H-pyrazol-4-ylamine.

¹H NMR (DMSO-d₆, 200 MHz) δ 11.7 (m, 1H), 9.27 (m, 1H), 7.86 (m, 1H), 3.64 (s, 3H), 2.04 (s, 3H) and 1.95 (s, 3H).

### (b) 1-(2,5-Dimethyl-3-furoyl)-1H-pyrazole-3-carboxylic acid (1,3,5-trimethyl-1H-pyrazol-4-yl)amide

The title compound was prepared as described in Example 1(d) from 1H-pyrazole-3-carboxylic acid (1,3,5-trimethyl-1H-pyrazol-4-yl)amide (see step (a) above) and 2,5-dimethyl-3-furoyl chloride.

MS (M⁺+H) m/z 342.

### Example 11

### 1-(1-Methyl-1H-pyrrol-2-yl)-1H-pyrazole-3-carboxylic acid (1,3,5-trimethyl-1H-pyrazol-4-yl)amide

The title compound was prepared as described in Example 1(d) from 1H-pyrazole-3-carboxylic acid (1,3,5-trimethyl-1H-pyrazol-4-yl)amide (see Example 10(a)) and 1-methyl-2-pyrroyl chloride.

MS (M⁺+H) m/z 327.

### Example 12

### 1-(3-Methyl-2-benzofuroyl)-1H-pyrazole-3-carboxylic acid (1,3,5-trimethyl-1H-pyrazol-4-yl)amide

The title compound was prepared as described in Example 1(d) from 1H-pyrazole-3-carboxylic acid (1,3,5-trimethyl-1H-pyrazol-4-yl)amide (see Example 10(a)) and 3-methyl-2-benzofuroyl chloride.

MS (M⁺+H) m/z 378.

### Example 13

### 1-(2-Fluoro-5-iodobenzoyl)-1H-pyrazole-3-carboxylic acid (1,3,5-trimethyl-1H-pyrazol-4-yl)amide

The title compound was prepared as described in Example 1(d) from 1H-pyrazole-3-carboxylic acid (1,3,5-trimethyl-1H-pyrazol-4-yl)amide (see Example 10(a)) and 2-fluoro-5-iodobenzoyl chloride.

MS (M⁺+H) m/z 468.

### Example 14

### 1-(2-Ethoxybenzoyl)-1H-pyrazole-3-carboxylic acid (3-methylisoxazol-5-yl)amide

### (a) 1H-Pyrazole-3-carboxylic acid (3-methylisoxazol-5-yl)amide

The title compound was prepared as described in Example 6(b) from dipyrazolo[1,5-a;1′,5′-d]pyrazine-4,9-dione (see Example 6(a)) and 3-methylisoxazol-5-ylamine.

¹H NMR (DMSO-d₆, 200 MHz) δ 7.85 (d, J=2 Hz, 1H), 6.86 (d, J=2 Hz, 1H), 6.24 (s, 1H), 2.2 (s, 3H).

### (b) 1-(2-Ethoxybenzoyl)-1H-pyrazole-3-carboxylic acid (3-methylisoxazol-5-yl)amide

The title compound was prepared as described in Example 1(d) from 1H-pyrazole-3-carboxylic acid (3-methylisoxazol-5-yl)amide (see step (a) above) and 2-ethoxybenzoyl chloride.

MS (4+H) m/z 341.

### Example 15

### 1-(3-Methyl-2-benzofuroyl)-1H-pyrazole-3-carboxylic acid (3-methylisoxazol-5-yl)amide

The title compound was prepared as described in Example 1(d) from 1H-pyrazole-3-carboxylic acid (3-methylisoxazol-5-yl)amide (see Example 14(a)) and 3-methyl-2-benzofuroyl chloride.

MS (M⁺+H) m/z 351.

### Example 16

### 1-(2-Furoyl)-1H-pyrazole-3-carboxylic acid (3-methylisoxazol-5-yl)amide

The title compound was prepared as described in Example 1(d) from 1H-pyrazole-3-carboxylic acid (3-methylisoxazol-5-yl)amide (see Example 14(a)) and 2-furoyl chloride.

MS (M⁺+H) m/z 287.

### Example 17

### 1-(1-Naphthoyl)-1H-pyrazole-3-carboxylic acid (3-methylisoxazol-5-yl)amide

The title compound was prepared as described in Example 1(d) from 1H-pyrazole-3-carboxylic acid (3-methylisoxazol-5-yl)amide (see Example 14(a)) and 1-naphthoyl chloride.

MS (M⁺+H) m/z 347.

### Example 18

### 4-{[1-(2,5-Dimethyl-3-furoyl)-1H-pyrazole-3-carbonyl]amino}benzoic acid methyl ester

### (a) 4-[(1H-Pyrazole-3-carbonyl)amino]benzoic acid methyl ester

Freshly prepared pyrazole-3-carbonyl chloride (5.80 g, 44.6 mmol; see Example 1(b)) was added to a solution of 4-aminobenzoic acid methyl ester (2.7 g, 17.9 mmol) in pyridine (50 mL). The solution was stirred at 85° C. for 3 h and concentrated. The residue was extracted twice with EtOAc and the combined extracts were washed with H₂O, dried with Na₂SO₄ and concentrated to dryness. The solid was washed with Et₂O to give the sub-title compound.

¹H NMR (CDCl₃, 200 MHz) δ 9.0 (s, 1H), 8.1 (d, J=6 Hz, 2H), 7.8 (d, J=6 Hz, 2H), 7.7 (s, J=2 Hz, 1H), 7.05 (s, J=2 Hz, 1H), 3.93 (s, 3H).

### (b) 4-{[1-(2,5-Dimethyl-3-furoyl)-1H-pyrazole-3-carbonyl]amino}benzoic acid methyl ester

The title compound was prepared as described in Example 1(d) from 4-[(1H-pyrazole-3-carbonyl)amino]benzoic acid methyl ester (see step (a) above) and 2,5-dimethyl-3-furoyl chloride.

MS (M⁺+H) m/z 368.

### Example 19

### 4-{[1-(5-Fluoro-2-methylbenzoyl)-1H-pyrazole-3-carbonyl]amino}benzoic acid methyl ester

The title compound was prepared as described in Example 1(d) from 4-[(1H-pyrazole-3-carbonyl)amino]benzoic acid methyl ester (see Example 18(a)) and 5-fluoro-2-methylbenzoyl chloride.

MS (M⁺+H) m/z 382.

### Example 20

### 4-{[1-(4-Pentylbenzoyl)-1H-pyrazole-3-carbonyl]amino}benzoic acid methyl ester

The title compound was prepared as described in Example 1(d) from 4-[(1H-pyrazole-3-carbonyl)amino]benzoic acid methyl ester (see Example 18(a)) and 4-pentylbenzoyl chloride.

MS (M⁺+H) m/z 420.

### Example 21

### 4-{[1-(2-Furoyl)-1H-pyrazole-3-carbonyl]amino}benzoic acid methyl ester

The title compound was prepared as described in Example 1(d) from 4-[(1H-pyrazole-3-carbonyl)amino]benzoic acid methyl ester (see Example 18(a)) and 2-furoyl chloride.

MS (M⁺+H) m/z 340.

### Example 22

### 1-(1-Methyl-1H-pyrrol-2-yl)-1H-pyrazole-3-carboxylic acid (4-dimethylaminophenyl)amide

### (a) 1H-Pyrazole-3-carboxylic acid (4-dimethylaminophenyl)amide

Dipyrazolo[1,5-a;1′,5′-d]pyrazine-4,9-dione (1.0 g, 5.3 mmol; see Example 6(a)) was added to a solution of dimethylaminophenylamine dihydrochloride (2.0 g; 11.6 mmol) and Et₃N (3.2 mL, 23.2 mmol) in DMF (20 mL). The mixture was stirred at 85° C. for 1 h and then concentrated. The residue was extracted twice with EtOAc and the combined extracts were washed with H₂O, dried with Na₂SO₄ and concentrated to dryness. The residue was purified by chromatography to afford the sub-title compound.

¹H NMR (CDCl₃, 200 MHz) δ 8.55 (s, 1H), 7.6 (d, J=2.5 Hz, 1H), 7.52 (d, J=9 Hz, 2H), 6.91 (d, J=2.5 Hz, 1H), 6.74 (s, J=9 Hz, 2H), 2.92 (s, 6H)

### (b) 1-(1-Methyl-1H-pyrrol-2-yl)-1H-pyrazole-3-carboxylic acid (4-dimethylaminophenyl)amide

The title compound was prepared as described in Example 1(d) from 1H-pyrazole-3-carboxylic acid (4-dimethylaminophenyl)amide (see step (a) above) and 1-methyl-2-pyrroyl chloride.

MS (M⁺+H) m/z 338.

### Example 23

### 1-(4-Pentylbenzoyl)-1H-pyrazole-3-carboxylic acid (4-dimethylaminophenyl)amide

The title compound was prepared as described in Example 1(d) from 1H-pyrazole-3-carboxylic acid (4-dimethylaminophenyl)amide (see Example 22(a)) and 4-pentylbenzoyl chloride.

MS (M⁺+H) m/z 405.

### Example 24

### 1-(3-Methyl-2-thienoyl)-1H-pyrazole-3-carboxylic acid (4-dimethylaminophenyl)amide

The title compound was prepared as described in Example 1(d) from 1H-pyrazole-3-carboxylic acid (4-dimethylaminophenyl)amide (see Example 22(a)) and 3-methyl-2-thienoyl chloride.

MS (M⁺+H) m/z 355.

### Example 25

### 1-(1-Naphthoyl)-1H-pyrazole-3-carboxylic acid (4-dimethylaminophenyl)amide

The title compound was prepared as described in Example 1(d) from 1H-pyrazole-3-carboxylic acid (4-dimethylaminophenyl)amide (see Example 22(a)) and 1-naphthoyl chloride.

MS (M⁺+H) m/z 385.

### Example 26

### 1-(2-Ethoxybenzoyl)-1H-pyrazole-3-carboxylic acid (5-chloro-2-cyanophenyl)amide

### (a) 1H-Pyrazole-3-carboxylic acid (5-chloro-2-cyanophenyl)amide

The title compound was prepared as described in Example 6(b) from dipyrazolo[1,5-a;1′,5′-d]pyrazine-4,9-dione (see Example 6(a)) and 5-chloro-2-cyanoaniline.

¹H NMR (CDCl₃, 200 MHz) δ 10.3 (s, 1H), 8.03 (m, 1H), 7.98 (m, 1H), 7.93 (d, J=8 Hz, 1H), 7.48 (2, J=8 Hz, 1H), 6.86 (m, 1H).

### (b) 1-(2-Ethoxybenzoyl)-1H-pyrazole-3-carboxylic acid (5-chloro-2-cyanophenyl)amide

The title compound was prepared as described in Example 1(d) from 1H-pyrazole-3-carboxylic acid (5-chloro-2-cyanophenyl)amide (see step (a) above) and 2-ethoxybenzoyl chloride.

MS (M⁺+H) m/z 395.

### Example 27

### 1-(5-Fluoro-2-methylbenzoyl)-1H-pyrazole-3-carboxylic acid (5-chloro-2-cyanophenyl)amide

The title compound was prepared as described in Example 1(d) from 1H-pyrazole-3-carboxylic acid (5-chloro-2-cyanophenyl)amide (see Example 26(a)) and 5-fluoro-2-methylbenzoyl chloride.

MS (M⁺+H) m/z 383.

### Example 28

### 1-(2-Furoyl)-1H-pyrazole-3-carboxylic acid (5-chloro-2-cyanophenyl)amide

The title compound was prepared as described in Example 1(d) from 1H-pyrazole-3-carboxylic acid (5-chloro-2-cyanophenyl)amide (see Example 26(a)) and 2-furoyl chloride.

MS (M⁺+H) m/z 341.

### Example 29

### 1-(1-Naphthoyl)-1H-pyrazole-3-carboxylic acid (5-chloro-2-cyanophenyl)amide

The title compound was prepared as described in Example 1(d) from 1H-pyrazole-3-carboxylic acid (5-chloro-2-cyanophenyl)amide (see Example 26(a)) and 1-naphthoyl chloride.

MS (M⁺+H) m/z 401.

### Example 30

### 1-Oleoyl-1H-pyrazole-3-carboxylic acid (2-chlorophenyl)amide

### (a) 1H-Pyrazole-3-carboxylic acid (2-chlorophenyl)amide

A mixture of dipyrazolo[1,5-a;1′,5′-d]pyrazine-4,9-dione (1.88 g, 10 mmol; see Example 6(a)), 2-chloroaniline (6.38 g, 50 mmol) and 4-(N,N-dimethylamino)pyridine (1.22 g, 10 mmol) was stirred at 120° C. for 30 min. After cooling to room temperature, EtOH (20 mL), followed by water (100 mL) and isohexane (100 mL) was added. The mixture was shaken for 10 min. The solid was filtered off, washed with 50% aqueous EtOH (20 mL), water (50 mL) and isohexane (50 mL), and dried in vacuum, to give the sub-title compound (yield: 2.45 g, 55%).

### (b) 1-Oleoyl-1H-pyrazole-3-carboxylic acid (2-chlorophenyl)amide

A mixture of 1H-pyrazole-3-carboxylic acid (2-chlorophenyl)amide (55 mg, 0.25 mmol; see step (a) above), oleoyl chloride (146 mg, 0.30 mmol) and 4-(N,N-dimethylamino)pyridine (36 mg, 0.30 mmol) in CH₂Cl₂ (5 mL) was stirred at 40° C. for 2 days. The mixture was diluted with CH₂Cl₂ (10 mL), washed with water (2×10 mL) and concentrated. Chromatographic purification gave the title compound (yield: 83 mg, 68%).

MS (M⁺+H) m/z 486.

¹H NMR (CDCl₃, 400 MHz) δ 9.41 (s, 1H), 8.56 (dd, J=8 Hz, 1 Hz, 1H), 8.30 (d, J=3 Hz, 1H), 7.42 (dd, J=8 Hz, 1 Hz, 1H), 7.33 (td, J=8 Hz, 1 Hz, 1H), 7.08 (td, J=8 Hz, 1 Hz, 1H), 7.03 (d, J=3 Hz, 1H), 5.36 (ddd, J=11 Hz, 6 Hz, 3 Hz, 1H), 5.32 ddd, J=41 Hz, 6 Hz, 3 Hz, 1H), 3.17 (t, J=8 Hz, 2H), 1.96-2.08 (m, 4H), 1.78-1.88 (m, 2H), 1.18-1.50 (m, 20H), 0.86 (t, J=7 Hz, 3H).

¹³C NMR (CDCl₃, 100.5 MHz) δ 172.03, 158.7, 150.2, 134.4, 130.3, 130.2, 129.8, 129.2, 128.0, 124.9, 123.0, 121.3, 109.5, 33.92, 32.0, 29.85, 29.77, 29.6, 29.4 (2C), 29.31, 29.25, 29.18, 27.3, 27.2, 24.6, 22.8, 14.2.

### Example 31

### 3-(2-Chlorophenylcarbamoyl)-1H-pyrazole-1-carboxylic acid (1R,2S,5R)-(−)-menthol ester

A mixture of 1H-pyrazole-3-carboxylic acid (2-chlorophenyl)amide (56 mg, 0.25 mmol; see Example 30(a)), (−)-menthyl chloroformate (82 mg, 0.38 mmol), and 4-(N,N-dimethylamino)pyridine (45 mg, 375 μmol) in dichloromethane (5 mL) was stirred at 40° C. for 16 h. The mixture was concentrated, dissolved in EtOAc, washed with water, and dried with MgSO₄. Chromatographic purification afforded the title product (yield: 69 mg, 69%).

MS (M⁺+H) m/z=404.

¹H NMR (CDCl₃, 400 MHz) δ 9.42 (s, 1H), 8.49 (dd, J=9, 2 Hz, 1H), 8.18 (d, J=3 Hz, 1H), 7.41 (dd, J=9, 2 Hz, 1H), 7.31 (dt, J=9, 2 Hz, 1H), 7.07 (dt, J=9, 2 Hz, 1H), 7.01 (d, J=3 Hz, 1H), 4.92 (dt, J=12, 4 Hz, 1H), 2.25 (m, 1H), 2.01 (dsept, J=8, 3 Hz, 1H), 1.80-1.70 (m, 2H), 1.70-1.50 (m, 2H), 1.29-1.08 (m, 2H), 0.95 (dd, J=7, 2 Hz, 7H), 0.84 (d, J=7 Hz, 3H).

¹³C NMR (100.5 MHz) δ 158.7, 150.5, 148.3, 134.3, 132.6, 129.2, 127.7, 124.8, 123.4, 121.5, 109.1, 80.5, 47.0, 40.4, 33.9, 31.4, 26.7, 23.8, 21.9, 20.5, 16.7.

### Example 32

### 3-(2-Chlorophenylcarbamoyl)-1H-pyrazole-1-carboxylic acid 2-chlorobenzyl ester

The title compound was prepared as described in Example 31 from 1H-pyrazole-3-carboxylic acid (2-chlorophenyl)amide (see Example 30(a)) and 2-chlorobenzyl chloroformate.

MS (M⁺+H) m/z 390.

¹H NMR (CDCl₃, 400 MHz) δ 9.44 (s, 1H), 8.49 (dd, J=8 Hz, 1 Hz, 1H), 8.21 (d, J=3 Hz, 1H), 7.59 (dd, J=7 Hz, 1H), 7.39-7.47 (m, 2H), 7.28-7.38 (m, 3H), 7.07 (td, J=8 Hz, 1 Hz, 1H), 7.02 (d, J=3 Hz, 1H), 5.62 (s, 2H).

¹³C NMR (100.5 MHz) δ 151.0, 148.5, 134.3, 134.0, 133.1, 131.9, 130.5, 130.4, 130.0, 129.3, 127.8, 127.2, 125.0, 123.5, 121.6, 109.5.

### Example 33

### 3-(2-Chlorophenylcarbamoyl)-1H-pyrazole-1-carboxylic acid 4-chlorophenyl ester

The title compound was prepared as described in Example 31 from 1H-pyrazole-3-carboxylic acid (2-chlorophenyl)amide (see Example 30(a)) and 4-chlorophenyl chloroformate.

MS (M⁺+H) m/z 376.

### Example 34

### 1H-Pyrazole-1,3-dicarboxylic acid 3-[(2-chlorophenyl)amide]1-dimethylamide

The title compound was prepared as described in Example 30(b) from 1H-pyrazole-3-carboxylic acid (2-chlorophenyl)amide (see Example 30(a)) and N,N-dimethylcarbamoyl chloride.

MS (M⁺+H) m/z 293.

### Example 35

### 1H-Pyrazole-1,3-dicarboxylic acid 1-[(3-chlorophenyl)amide]3-[(2-chlorophenyl)amide]

A mixture of 1H-pyrazole-3-carboxylic acid (2-chlorophenyl)amide (55 mg, 0.25 mmol; see Example 30(a)), 3-chlorophenylisocyanate (46 mg, 0.30 mmol) and toluene (5 mL) was stirred at 100° C. for 18 h. The solution was concentrated and isohexane (10 mL) was added. The title compound was filtered off and washed with isohexane (10 mL) (yield: 65 mg, 69%).

MS (M⁺+H) m/z 375.

¹H NMR (C₆D₆, 400 MHz) δ 9.05 (s, 1H), 8.91 (d, J=8 Hz, 1H), 8.31 (s, 1H), 7.72 (d, J=3 Hz, 1H), 7.61 (s, 1H), 7.07 (d, J=8 Hz, 1H), 6.94 (t, J=8 Hz, 1H), 6.85 (d, J=8 Hz, 1H), 6.72 (t, J=8 Hz, 1H), 6.69 (d, J=3 Hz, 1H), 6.58 (t, J=8 Hz, 1H).

¹³C NMR (100.5 MHz) δ 158.1, 149.1, 145.3, 137.5, 135.0, 134.7, 130.6, 130.1, 129.0, 128.4, 124.9, 124.7, 122.5, 121.3, 119.6, 117.5, 109.4.

### Example 36

### 1H-Pyrazole-1,3-dicarboxylic acid 3-[(2-chlorophenyl)amide]1-[(3-fluorophenyl)amide]

The title compound was prepared as described in Example 35 from 1H-pyrazole-3-carboxylic acid (2-chlorophenyl)amide (see Example 30(a)) and 3-fluorophenylisocyanate.

MS (M⁺+H) m/z 359.

### Example 37

### 1H-Pyrazole-1,3-dicarboxylic acid 3-[(2-chlorophenyl)amide]1-[(3-trifluoromethylphenyl)amide]

The title compound was prepared as described in Example 35 from 1H-pyrazole-3-carboxylic acid (2-chlorophenyl)amide (see Example 30(a)) and 3-trifluoromethylphenylisocyanate.

MS (M⁺+H) m/z 409.

### Example 38

### 1H-Pyrazole-1,3-dicarboxylic acid 3-[(2-chlorophenyl)amide]1-[(4-nitrophenyl)amide]

The title compound was prepared as described in Example 35 from 1H-pyrazole-3-carboxylic acid (2-chlorophenyl)amide (see Example 30(a)) and 4-nitrophenylisocyanate.

MS (M⁺+H) m/z 386.

### Example 39

### 1-(Octane-1-sulfonyl)-1H-pyrazole-3-carboxylic acid (2-chlorophenyl)amide

The title compound was prepared as described in Example 30(b) from 1H-pyrazole-3-carboxylic acid (2-chlorophenyl)amide (see Example 30(a)) and 1-octanesulfonyl chloride.

MS (M⁺+H) m/z 398.

### Example 40

### 1-(3-Chlorobenzenesulfonyl)-1H-pyrazole-3-carboxylic acid (2-chlorophenyl)amide

The title compound was prepared as described in Example 30(b) from 1H-pyrazole-3-carboxylic acid (2-chlorophenyl)amide (see Example 30(a)) and 3-chlorobenzenesulfonyl chloride.

MS (M⁺+H) m/z 396.

¹H NMR (CDCl₃, 400 MHz) δ 9.25 (s, 1H), 8.45 (dd, J=8 Hz, 1 Hz, 1H), 8.17 (d, J=3 Hz, 1H), 8.09 (t, J=2 Hz, 1H), 7.97 (dt, J=8 Hz, 1 Hz, 1H), 7.68 (ddd, J=8 Hz, 2 Hz, 1 Hz, 1H), 7.54 (t, J=8 Hz, 1H), 7.41 (dd, J=8 Hz, 1 Hz, 1H), 7.29 (td, J=8 Hz, 1 Hz, 1H), 7.07 (td, J=8 Hz, 1 Hz, 1H), 7.00 (d, J=3 Hz, 1H).

¹³C NMR (100.5 MHz) δ 152.0, 137.8, 136.0, 135.5, 134.1, 133.1, 131.0, 129.3, 128.7, 127.9, 126.6, 125.1, 123.3, 121.4, 121.3, 109.2.

### Example 41

### 1-(4-tert-Butylbenzenesulfonyl)-1H-pyrazole-3-carboxylic acid (2-chlorophenyl)amide

The title compound was prepared as described in Example 30(b) from 1H-pyrazole-3-carboxylic acid (2-chlorophenyl)amide (see Example 30(a)) and 4-tert-butylbenzenesulfonyl chloride.

MS (M⁺+H) m/z 418.

¹H NMR (CDCl₆, 400 MHz) δ 9.27 (s, 1H), 8.45 (dd, J=8 Hz, 1 Hz, 1H), 8.16 (d, J=3 Hz, 1H), 7.98-8.02 (m, 2H), 7.56-7.61 (m, 2H), 7.40 (dd, J=8 Hz, 1 Hz, 1H), 7.29 (td, J=8 Hz, 1 Hz, 1H), 7.07 (td, J=8 Hz, 1 Hz, 1H), 6.96 (d, J=3 Hz, 1H), 1.33 (s, 9H).

¹³C NMR (100.5 MHz) δ 159.6, 158.3, 151.3, 134.3, 133.1, 132.9, 129.3, 128.6 (2C), 127.9, 126.8 (2C), 125.0, 123.2, 121.4, 108.8, 35.6, 31.0 (3C).

### Example 42

### 5-[3-(2-Chlorophenylcarbamoyl-1H-pyrazole-1-sulfonyl]furan-2-carboxylic acid methyl ester

The title compound was prepared as described in Example 30(b) from 1H-pyrazole-3-carboxylic acid (2-chlorophenyl)amide (see Example 30(a)) and 5-carbomethoxy-2-furansulfonyl chloride.

MS (M⁺+H) m/z 410.

### Example 43

### 1-(3-Chlorobenzoyl)-1H-pyrazole-3-carboxylic acid (2-chlorophenyl)methylamide

### (a) 1H-Pyrazole-3-carboxylic acid (2-chlorophenyl)methylamide

The title compound was prepared as described in Example 30(a) from dipyrazolo[1,5-a;1′,5′-d]pyrazine-4,9-dione (see Example 6(a)) and 2-chloro-N-methylaniline.

MS (M⁺+H) m/z 236.

### (b) 1-(3-Chlorobenzoyl)-1H-pyrazole-3-carboxylic acid (2-chlorophenyl)methylamide

The title compound was prepared as described in Example 30(b) from 1H-pyrazole-3-carboxylic acid (2-chlorophenyl)methylamide (see step (a) above) and 3-chlorobenzoyl chloride.

MS (M⁺+H) m/z 374.

### Example 44

### 1H-Pyrazole-1,3-dicarboxylic acid 1-[(3-chloro-phenyl)amide]3-[(2-chlorophenyl)methylamide]

The title compound was prepared as described in Example 35 from 1H-pyrazole-3-carboxylic acid (2-chlorophenyl)methylamide (see Example 43(a)) and 3-chlorophenylisocyanate.

MS (M⁺+H) m/z 389.

### Example 45

### 3-[(2-Chlorophenyl)methylcarbamoyl]-1H-pyrazole-1-carboxylic acid 2-chlorobenzyl ester

The title compound was prepared as described in Example 31 from 1H-pyrazole-3-carboxylic acid (2-chlorophenyl)methylamide (see Example 43(a)) and 2-chlorobenzyl chloroformate.

MS (M⁺+H) m/z 404.

### Example 46

### 1-(3-Chlorobenzenesulfonyl)-1H-pyrazole-3-carboxylic acid (2-chlorophenyl)methylamide

The title compound was prepared as described in Example 30(b) from 1H-pyrazole-3-carboxylic acid (2-chlorophenyl)methylamide (see Example 43(a)) and 3-chlorobenzenesulfonyl chloride.

MS (M⁺+H) m/z 410.

### Example 47

### 1-Phenylthiocarbamoyl-1H-pyrazole-3-carboxylic acid (2-chlorophenyl)amide

A mixture of 1H-pyrazole-3-carboxylic acid (2-chlorophenyl)amide (10 mg, 44.5 μmol; see Example 43(a)), phenylisothiocyanate (30 mg, 0.22 mmol) and anhydrous K₂CO₃ (69 mg, 0.5 mmol) in dry acetone (2 mL) was stirred for 5 h at room temperature. The reaction mixture was then filtered and concentrated. Purification by chromatography gave the title compound (yield: 12 mg, 76%) as a white solid.

MS (M⁺+H) m/z 357.

¹H NMR (400 MHz) δ 10.52 (s, 1H), 9.22 (s, 1H), 8.84 (d, J=3 Hz, 1H), 8.55 (dd, J=9, 2 Hz, 1H), 7.80 (m [app d, J=8 Hz], 2H), 7.50 (m [app t, J=8 Hz], 2H), 7.43 (dd, J=9, 2 Hz, 1H), 7.35 (m [app dq, J=7, 1 Hz], 2H), 7.11 (dt, J=8,1 Hz, 1H), 7.06 (d, J=3 Hz, 1H).

¹³C NMR (100.5 MHz) δ 172.0, 158.3, 148.7, 136.7, 134.1, 133.2, 129.23, 129.16, 128.0, 127.4, 125.0, 123.6, 122.9, 121.4, 110.0.

### Example 48

### N-(2-Chloro-4-fluorophenyl)-1-(1-methylpiperazine-4-carbonyl-1H-pyrazole-3-carboxamide

### (a) N-(2-Chloro-4-fluorophenyl)-1H-pyrazole-3-carboxamide

The sub-title compound was prepared according to the procedure described above in Example 30(a) using dipyrazolo[1,5-a;1′,5′-d]pyrazine-4,9-dione (see Example 6(a)) and 2-chloro-4-fluoroaniline.

MS (M⁺) m/z=239.

¹H NMR (DMSO-D₆, 300 MHz) δ 13.48 (broad s, 1H), 9.67 (s, 1H), 7.99 (m, 1H), 7.91 (d, 1H), 7.56 (dd, 1H), 7.28 (ddd, 1H), 6.81 (d, 1H).

### (b) N-(2-Chloro-4-fluorophenyl -1-(1-methylpiperazine-4-carbonyl)-1H-pyrazole-3-carboxamide

N-(2-Chloro-4-fluorophenyl)-1H-pyrazole-3-carboxamide (50 mg, 209 μmol, 1 equiv.; see step (a) above) was mixed with the hydrochloride salt of 4-methyl-piperazine-1-carbonyl chloride (83 mg, 410 μmol, 2 equiv.) and DMAP (50 mg, 410 μmol, 2 equiv.) in toluene (5 mL) at 90° C. The reaction mixture was left stirring for 16 h and then cooled. The precipitate from the reaction mixture was collected and recrystallised from EtOH/EtOAc to yield the hydrochloride salt of the title compound (a white powder). 0.5 M NaOH (aq.) and ethyl acetate were added to the salt. The organic phase was dried and concentrated to afford the free base of the title compound as an off-white solid.

MS: m/z=366 (M⁺+1).

¹H NMR (CDCl₃): δ 9.21 (br s, 1H), 8.56 (m, 1H), 8.17 (d, J=3 Hz, 1H), 7.18 (m, 1H), 7.07 (m, 1H), 6.98 (d, J=3 Hz, 1H), 3.99 (m, 4H), 2.68 (m, 4H), 2.44 (s, 3H).

### Example 49

### N³-(2-Chlorophenyl)-N¹-(1-(2,2,2-trifluoroacetyl)piperidin-4-yl)-1H-pyrazole-1,3-dicarboxamide

1H-Pyrazole-3-carboxylic acid (2-chlorophenyl)amide (50 mg, 226 μmol, 1 equiv.; see Example 30(a)), 2,2,2-trifluoro-1-(4-isocyanatopiperidin-1-yl)ethanone (76 mg, 338 μmol, 1.5 equiv.) and DMAP (33 mg, 270 μmol, 1.2 equiv.) were added to toluene at room temperature. The reaction was left stirring for 24 h, after which the excess solvent removed. Purification by chromatography afforded the title compound.

MS: m/z=444 (M⁺+1).

¹H NMR (dmso-d₆): δ 9.86 (s, 1H), 8.52 (d, J=8 Hz, 1H), 8.45 (d, J=3 Hz, 1H), 7.76 (dd, J=8, 1 Hz, 1H), 7.60 (dd, J=8, 1 Hz, 1H), 7.42 (dt, J=8, 1 Hz, 1H), 7.31 (dt, J=8 Hz, 1H); 6.99 (d, J=3 Hz, 1H), 4.31 (m, 1H), 4.12-4.02 (m, 1H), 3.91 (m, 1H), 3.43 (t, J=12 Hz, 1H), 3.08 (t, J=12 Hz, 1H), 2.09-1.99 (m, 2H), 1.71-1.56 (m, 2H).

### Example 50

### Pyrazole-1,3-dicarboxylic acid-1-[(3-chlorophenyl)amide]-3-(methylphenylamide)

### (a) 1H-Pyrazole-3-carboxylic acid methylphenylamide

A mixture of dipyrazolo[1,5-a;1′,5′-d]pyrazine-4,9-dione (1.00 g, 5.30 mmol; see Example 6(a)), N-methyl aniline (5.00 g, 0.38 mmol), and 4-(N,N-dimethylamino)pyridine (0.650 g, 5.30 mmol) was stirred at 120° C. for 4 h. After cooling to room temperature, EtOH (15 ml), water (50 ml) and iso-hexane (50 ml) were added. The resulting mixture was shaken and then the aqueous phase was separated. The organic phase was concentrated to afford the crude product (1.27 g). Purification by recrystallisation from EtOH afforded the sub-title compound (yield: 0.990 g, 47%)

MS (M⁺+H) m/z=202.

¹H NMR (DMSO-D₆, 400 MHz) δ 7.60 (s, 1H), 7.55-7.10 (m, 5H), 6.15 (s, 1H), 3.35 (s, 3H).

### (b) Pyrazole-1,3-dicarboxylic acid-1-[(3-chlorophenyl)amide]-3-(methylphenylamide)

A mixture of 1H-pyrazole-3-carboxylic acid methylphenylamide (0.200 g, 0.994 mmol; see step (a) above), 1-chloro-3-isocyanatobenzene (0.183 g, 1.19 mmol) and toluene (5 ml) was stirred at 100° C. for 18 h. The reaction mixture was then cooled to room temperature and the solvent was evaporated. iso-Hexane (10 ml) was then added and the resulting precipitate was filtered off. The precipitate was then washed with isohexane (20 ml) and then dried under reduced pressure to give the title compound (yield: 0.212 g, 60%).

MS (M⁺+H) m/z=356.

¹H NMR (CDCl₃, 400 MHz) δ 8.45 (s, 1H), 8.05 (s, 1H), 7.60-7.00 (m, 9H), 6.35 (s, 1H), 3.50 (s, 3H).

### Example 51

### 1-(4-tert-Butylbenzenesulfonyl)-1H-pyrazole-3-carboxylic acid methylphenylamide

A mixture of 1H-pyrazole-3-carboxylic acid methylphenylamide (0.200 g, 0.994 mmol; see Example 50(a)), 4-tert-butylbenzenesulfonyl chloride (0.278 g, 1.19 mmol), 4-(N,N-dimethylamino)pyridine (0.146 g, 1.19 mmol) and dichloromethane (5 ml) was stirred at 40° C. for 48 h. The reaction mixture was then cooled to room temperature, dichloromethane (10 ml) was added and the mixture was washed twice with water (10 ml). The organic phase was filtered through a short silica plug (eluting with acetone) and concentrated. The crude product was purified by recrystallisation from hexane to afford the title compound (yield: 0.257 g, 65%).

MS (M⁺+H) m/z=399.

¹H NMR (CDCl₃, 400 MHz) δ 7.87 (s, 1H), 7.73-7.58 (m, 2H), 7.53-7.41 (m, 2H), 7.25-6.95 (m, 5), 6.38 (s, 1H), 3.40 (s, 3H), 1.29 (s, 9H).

¹³C NMR (CDCl₃, 100.5 MHz) δ 162.4, 158.7, 151.8, 144.0, 133.5, 131.0, 129.1, 128.2, 127.3, 126.5, 110.0, 38.4, 35.5, 31.0.

### Example 52

### 1-(3-Chlorobenzenesulfonyl)-1H-pyrazole-3-carboxylic acid methylphenylamide

A mixture of 1H-pyrazole-3-carboxylic acid methylphenylamide (0.100 g, 0.497 mmol; see Example 50(a)), 3-chlorobenzenesulfonyl chloride (0.126 g, 0.596 mmol), 4-(N,N-dimethylamino)pyridine (0.073 g, 0.596 mmol) and dichloromethane (5 ml) was stirred at 40° C. for 48 h. The reaction mixture was then cooled to room temperature, dichloromethane (20 ml) was added and the mixture was washed twice with water (10 ml). The organic phase was filtered through a short silica plug (eluting with acetone) and concentrated. The crude product was purified by recrystallisation from isohexane to afford the title compound (yield: 0.140 g, 75%) as white crystals.

MS (M⁺+H) m/z=376.

¹H NMR (CDCl₃, 400 MHz) δ 7.92 (s, 1H), 7.74 (s, 1H), 7.67-7.50 (m, 2H), 7.48-7.34 (m, 1H), 7.33-6.92 (m, 5H), 6.50 (s, 1H), 3.50 (s, 3H).

¹³C NMR (CDCl₃, 100.5 MHz) δ 162.0, 152.4, 143.9, 138.1, 135.5, 134.9, 131.2, 130.7, 129.1, 128.3, 127.5, 127.2, 126.5, 110.5, 38.4.

### Example 53

### 1-(3-Chlorobenzoyl)-1H-pyrazole-3-carboxylic acid methylphenylamide

A mixture of 1H-pyrazole-3-carboxylic acid methylphenylamide (0.100 g, 0.497 mmol; see Example 50(a)), 3-chlorobenzoyl chloride (0.104 g, 0.596 mmol), 4-(N,N-dimethylamino)pyridine (0.073 g, 0.596 mmol) and dichloromethane (5 ml) was stirred at 40° C. for 48 h. The reaction mixture was then cooled to room temperature, dichloromethane (10 ml) was added and the mixture was washed twice with water (10 ml). The organic phase was filtered through a short silica plug (eluting with acetone) and concentrated. The crude product was purified by recrystallisation from isohexane, benzene and diethyl ether to afford the title compound (yield: 0.025 g, 15%) as white crystals.

MS (M⁺+H) m/z=340.

¹H NMR (CDCl₃, 400 MHz) δ 8.26 (s, 1H), 7.84 (s, 1H), 7.58-7.10 (m, 8H), 6.65 (s, 1H), 3.50 (s, 3H).

### Example 54

### 3-(Methylphenylcarbamoyl)pyrazole-1-carboxylic acid 2-chlorobenzyl ester

A mixture of 1H-pyrazole-3-carboxylic acid methylphenylamide (0.100 g, 0.497 mmol; see Example 50(a)), 2-chlorobenzyl chloroformate (0.122 g, 0.596 mmol), 4-(N,N-dimethylamino)pyridine (0.073 g, 0.596 mmol) and dichloromethane (5 ml) was stirred at 40° C. for 48 h. The reaction mixture was then cooled to room temperature, dichloromethane (10 ml) was added and the mixture was washed twice with water (10 ml). The organic phase was separated and concentrated. The crude product was then purified by recrystallisation from iso-hexane, then from MeOH and finally from benzene to afford the title compound (yield: 0.050 g, 27%) as white crystals.

MS (M⁺+H) m/z=370.

¹H NMR (CDCl₃, 400 MHz) δ 7.90 (s, 1H), 7.55-7.05 (m, 9H), 6.20 (s, 1H), 5.48 (s, 2H), 3.50 (s, 3H).

Anal. calcd for C₁₉H₁₆ClN₃O₃: C, 61.71; H, 4.36; Found: C, 61.29: H, 4.66.

### Example 55

### Pyrazole-1,3-dicarboxylic acid 1-butylamide 3-[(2-chlorophenyl)methylamide]

A mixture of 1H-pyrazole-3-carboxylic acid (2-chlorophenyl)methylamide (0.052 g, 0.220 mmol; see Example 43(a)), 1-isocyanatobutane (0.026 g, 0.265 mmol), and toluene (5 ml) was stirred at 100° C. for 20 h. The reaction mixture was then cooled to room temperature. The solvent was then evaporated, diethyl ether (30 ml) was added and the resulting precipitate was filtered off. The mother liquor was evaporated to afford the title compound (yield: 0.054 g, 73%) as a thick oil.

MS (M⁺+H) m/z=335.

### Example 56

### Pyrazole-1,3-dicarboxylic acid 1-benzylamide 3-[(2-chlorophenyl)methylamide]

A mixture of 1H-pyrazole-3-carboxylic acid (2-chlorophenyl)methylamide (0.052 g, 0.220 mmol; see Example 43(a)), isocyanatomethylbenzene (0.036 g, 0.270 mmol), and toluene (5 ml) was stirred at 100° C. for 24 h.

The reaction mixture was then cooled to room temperature, filtered, concentrated and then dried under high vacuum for three days to give the pure title compound (yield: 0.048 g, 58%).

MS (M⁺+H) m/z=369.

¹H NMR (CDCl₃, 400 MHz) δ 7.98 (m, 1H), 7.48-7.10 (m, 7H), 7.08-7.0 (m, 1H), 6.91-6.83 (m, 1H), 6.61-6.55 (m, 1H), 6.45 (br s, 1H), 4.50-4.30 (m, 2H), 3.35 (s, 3H);

¹³C NMR (CDCl₃, 100.5 MHz) δ 161.9, 149.0, 148.4, 142.0, 136.9, 133.2, 130.1, 130.0, 129.2, 128.9, 128.6, 128.5, 128.2, 127.5, 110.4, 44.5, 37.1.

### Example 57

### Pyrazole-1,3-dicarboxylic acid 1-(benzo[1,3]dioxol-5-ylamide) 3-(2-chloropyridin-3-yl)amide

### (a) 1H-Pyrazole-3-carboxylic acid(2-chloropyridin-3-yl)amide

A mixture of dipyrazolo[1,5-a;1′,5′-d]pyrazine-4,9-dione (2.66 mmol, 500 mg, see Example 6(a)), 3-amino-2-chloropyridine (6.64 mmol, 854 mg), and DMAP (1.33 mmol, 162 mg) was heated at 190° C. for 5 minutes using microwaves with a Smith synthesizer from Personal Chemistry. The reaction mixture was allowed to cool and mixed with water (10 mL) and EtOAc (10 mL). The organic layer was separated and the aqueous layer was extracted with ethyl acetate (3×5 mL). The combined organic phases were concentrated and purified by preparative HPLC to give 139 mg of the sub-title compound as a white solid.

MS (M⁺) m/z=222.

¹H NMR (DMSO-D₆, 300 MHz) δ 13.63 (broad s, 1H), 9.60 (broad s, 1H), 8.48 (d, 1H), 8.22 (dd, 1H), 7.95 (d, 1H), 7.50 (dd, 1H), 6.83 (d, 1H).

(b) Pyrazole-1,3-dicarboxylic acid 1-(benzo[1,3]dioxol-5-ylamide) 3-(2-chloro-pyridin-3-yl)amide

A mixture of 1H-pyrazole-3-carboxylic acid (2-chloropyridin-3-yl)amide (0.25 mmol, 55 mg; see step (a) above), 3,4-methylenedioxyphenyl isocyanate (0.30 mmol, 49 mg) and toluene (0.5 mL) was heated at 180° C. for 10 min using microwaves with a Smith synthesizer from Personal Chemistry. The title compound was precipitated by addition of isohexane (4 mL) and collected by filtration (yield 34 mg).

MS (M⁺) m/z=385.

¹H NMR (CDCl₃, 300 MHz) δ 9.22 (s, 1H), 8.92 (dd, 1H), 8.79 (s, 1H), 8.43 (d, 1H), 8.20 (dd, 1H), 7.37 (dd, 1H), 7.31 (d, 1H), 7.10 (d, 1H), 6.95 (dd, 1H), 6.86 (d, 1H), 6.04 (s, 2H).

### Example 58

### 6-{[3-(2-Chloropyridin-3-ylcarbamoyl)pyrazole-1-carbonyl]-amino}hexanoic acid ethyl ester

The title compound was prepared according to the procedure described above in Example 57(b) from 1H-pyrazole-3-carboxylic acid (2-chloropyridin-3-yl)amide (see Example 57(a)) and ethyl 6-isocyanatohexanoate.

MS (M⁺) m/z=407.

¹H NMR (CDCl₃, 300 MHz) δ 9.19 (s, 1H), 8.88 (dd, 1H), 8.33 (d, 1H), 8.18 (dd, 1H), 7.35 (ddd, 1H), 7.10 (t, 1H), 7.02 (d, 1H), 4.12 (q, 2H), 3.52 (q, 2H), 2.35 (t, 2H), 1.80-1.66 (m, 4H), 1.54-1.42 (m, 2H), 1.25 (t, 3H).

### Example 59

### Pyrazole-1,3-dicarboxylic acid 3-(2-chloropyridin-3-yl)amide) 1-pentylamide

The title compound was prepared according to the procedure described above in Example 57(b) from 1H-pyrazole-3-carboxylic acid (2-chloropyridin-3-yl)amide (see Example 57(a)) and pentyl isocyanate.

MS (M⁺) m/z=335.

¹H NMR (CDCl₃, 300 MHz) δ 9.17 (s, 1H), 8.89 (dd, 1H), 8.32 (d, 1H), 8.17 (dd, 1H), 7.33 (dd, 1H), 7.05 (t, 1H), 7.01 (d, 1H), 3.50 (q, 2H), 1.76-1.64 (m, 2H), 1.46-1.37 (m, 4H), 0.99-0.90 (m, 3H).

### Example 60

### Pyrazole-1,3-dicarboxylic acid 1-benzo[1,3]dioxol-5-ylamide 3-(2-fluoro-5-trifluoromethylphenylamide)

### (a) 1H-Pyrazole-3-carboxylic acid 2-fluoro-5-trifluoromethylphenylamide

The sub-title compound was prepared according to the procedure described above in Example 57(a) using dipyrazolo[1,5-a;1′,5′-d]pyrazine-4,9-dione (see Example 6(a)) and 2-fluoro-5-(trifluoromethyl)aniline.

MS (M⁺) m/z=273.

¹H NMR (DMSO-D₆, 300 MHz) δ 12.94 (broad s, 1H), 9.45 (broad s, 1H), 8.26 (dd, 1H), 7.91 (d, 1H), 7.62 (dd, 1H), 7.55 (dd, 1H), 6.83 (d, 1H).

### (b) Pyrazole-1,3-dicarboxylic acid 1-benzo[1,3]dioxol-5-ylamide 3-(2-fluoro-5-trifluoromethylphenylamide)

The title compound was prepared according to the procedure described above in Example 57(b) from 1H-pyrazole-3-carboxylic acid 2-fluoro-5-trifluoromethylphenylamide (see step (a) above) and 3,4-(methylenedioxy)phenyl isocyanate.

MS (M⁺) m/z=436.

¹H NMR (DMSO-D₆, 300 MHz) δ 10.43 (broad s, 1H), 10.17 (broad s, 1H), 8.55 (d, 1H), 8.29 (dd, 1H), 7.70 (ddd, 1H), 7.60 (dd, 1H), 7.31 (d, 1H), 7.12 (dd, 1H), 7.06 (d, 1H), 6.98 (d, 1H), 6.06 (s, 2H).

### Example 61

### Pyrazole-1,3-dicarboxylic acid 3-(2-fluoro-5-trifluoromethylphenylamide) 1-(3-trifluoromethylphenylamide)

The title compound was prepared according to the procedure described above in Example 57(b) from 1H-pyrazole-3-carboxylic acid 2-fluoro-5-trifluoromethylphenylamide (see Example 60(a)) and 3-trifluoromethylphenyl isocyanate.

MS (M⁺) m/z=460.

¹H NMR (CDCl₃, 400 MHz) δ 9.00 (s, 1H), 8.90-8.86 (m, 2H), 8.44 (d, 1H), 7.96 (dd, 1H), 7.88 (dd, 1H), 7.59 (dd, 1H), 7.51 (ddd, 1H), 7.43 (dddd, 1H), 7.30 (ddd, 1H), 7.13 (d, 1H).

### Example 62

### Pyrazole-1,3-dicarboxylic acid 3-(3-cyanophenylamide) 1-(4-trifluoromethoxyphenylamide)

### (a) 1H-Pyrazole-3-carboxylic acid 3-cyanophenylamide

The sub-title compound was prepared according to the procedure described above in Example 57(a) using dipyrazolo[1,5-a;1′,5′-d]pyrazine-4,9-dione (see Example 6(a)) and 3-cyanoaniline.

MS (M⁺) 171/z=212.

¹H NMR (DMSO-D₆, 300 MHz) δ 13.53 (s, 1H), 10.45 (s, 1H), 8.28 (s, 1H), 8.10 (dd, 1H), 7.90 (s, 1H), 7.50-7.60 (m, 2H), 6.80 (s, 1H).

### (b) Pyrazole-1,3-dicarboxylic acid 3-(3-cyanophenylamide) 1-(4-trifluoromethoxyphenylamide)

The title compound was prepared according to the procedure described above in Example 57(b) from 1H-pyrazole-3-carboxylic acid 3-cyanophenylamide (see step (a) above) and 4-(trifluoromethoxy)phenyl isocyanate.

MS (M⁺) m/z=415.

¹H NMR (CDCl₃, 300 MHz) δ 9.00 (s, 1H), 8.75 (s, 1H), 8.43 (s, 1H), 8.10-8.03 (m, 2H), 7.77-7.70 (m, 2H), 7.53 (dd, 1H), 7.46 (ddd, 1H), 7.35-7.29 (m, 2H), 7.12 (d, 1H).

### Example 63

### Pyrazole-1,3-dicarboxylic acid 3-(2-chloro-4-fluorophenylamide) 1-(4-trifluoromethoxyphenylamide)

The title compound was prepared according to the procedure described above in Example 57(b) from 1H-pyrazole-3-carboxylic acid 2-chloro-4-fluorophenylamide (see Example 48(a)) and 4-(trifluoromethoxy)phenyl isocyanate.

MS (M⁺) m/z=442.

¹H NMR (CDCl₃, 300 MHz) δ 9.07 (s, 1H), 8.95 (s, 1H), 8.60-8.34 (m, 2H), 7.70-7.60 (m, 2H), 7.40-7.18 (m, 3H), 7.17-7.00 (m, 2H).

### Example 64

### Pyrazole-1,3-dicarboxylic acid 1-(benzo[13]dioxol-5-ylamide) 3-(2-chloro-4-fluorophenylamide

The title compound was prepared according to the procedure described above in Example 57(b) from 1H-pyrazole-3-carboxylic acid 2-chloro-4-fluorophenylamide (see Example 48(a)) and 3,4-(methylenedioxy)phenyl isocyanate.

MS (M⁺) m/z=402.

¹H NMR (CDCl₃, 300 MHz) δ 9.09 (s, 1H), 8.79 (s, 1H), 8.52 (dd, 1H), 8.41 (d, 1H), 7.30 (d, 1H), 7.23 (dd, 1H), 7.09 (ddd, 1H), 7.08 (d, 1H), 6.95 (dd, 1H), 6.86 (d, 1H), 6.04 (s, 2H).

### Example 65

### 6-{[3-(2-Chloro-4-fluorophenylcarbamoyl)pyrazole-1-carbonyl]-amino}-hexanoic acid ethyl ester

The title compound was prepared according to the procedure described above in Example 57(b) from 1H-pyrazole-3-carboxylic acid 2-chloro-4-fluorophenylamide (see Example 48(a)) and ethyl 6-isocyanatohexanoate.

MS (M⁺) m/z=424.

¹H NMR (CDCl₃, 300 MHz) δ 9.06 (s, 1H), 8.48 (dd, 1H), 8.32 (d, 1H), 7.21 (dd, 1H), 7.10 (t, 1H), 7.08 (ddd, 1H), 7.02 (d, 1H), 4.12 (q, 2H), 3.51 (q, 2H), 2.35 (t, 2H), 1.79-1.65 (m, 4H), 1.53-1.41 (m, 2H), 1.25 (t, 3H).

### Example 66

### Pyrazole-1,3-dicarboxylic acid 3-(2-chloro-4-fluorophenylamide) 1-pentylamide

The title compound was prepared according to the procedure described above in Example 57(b) from 1H-pyrazole-3-carboxylic acid 2-chloro-4-fluorophenylamide (see Example 48 (a)) and pentyl isocyanate.

MS (M⁺) m/z=352.

¹H NMR (CDCl₃, 300 MHz) δ 9.08 (s, 1H), 8.51 (dd, 1H), 8.32 (d, 1H), 7.21 (dd, 1H), 7.13-7.00 (m, 1H), 3.50 (q, 2H), 1.78-1.65 (m, 2H), 1.47-1.38 (m, 4H), 1.00-0.92 (m, 3H).

### Example 67

### Pyrazole-1,3-dicarboxylic acid 3-(2,6-dichlorophenylamide) 1-(4-trifluoromethoxyphenylamide

### (a) 1H-Pyrazole-3-carboxylic acid 2,6-dichloro-phenylamide

The sub-title compound was prepared according to the procedure described above in Example 57(a) using dipyrazolo[1,5-a;1′,5′-d]pyrazine-4,9-dione (see Example 6(a)) and 2,6-dichloroaniline.

MS (M⁺) m/z=255.

¹H NMR (DMSO-D₆, 300 MHz) δ 13.45 (broad s, 1H), 10.03 (broad s, 1H), 7.87 (s, 1H), 7.56 (d, 2H), 7.37 (t, 1H), 6.77 (s, 1H).

### (b) Pyrazole-1,3-dicarboxylic acid 3-(2,6-dichlorophenylamide) 1-(4-trifluoro-methoxyphenylamide)

The title compound was prepared according to the procedure described above in Example 57(b) from 1H-pyrazole-3-carboxylic acid 2,6-dichlorophenylamide (see step (a) above) and 4-(trifluoromethoxy)phenyl isocyanate.

MS (M⁺) m/z=458.

¹H NMR (CDCl₃, 300 MHz) δ 9.01 (s, 1H), 8.41 (d, 1H), 8.36 (s, 1H), 7.74-7.68 (m, 2H), 7.47-7.42 (m, 2H), 7.33-7.26 (m, 3H), 7.12 (d, 1H).

### Example 68

### Pyrazole-1,3-dicarboxylic acid 3-(2-bromo-4-trifluoromethoxyphenylamide) 1-(3-trifluoromethylphenylamide)

### (a) 1H-Pyrazole-3-carboxylic acid 2-bromo-4-trifluoromethoxyphenylamide

The sub-title compound was prepared according to the procedure described above in Example 57(a) using dipyrazolo[1,5-a;1′,5′-d]pyrazine-4,9-dione (see Example 6(a)) and 2-bromo-4-trifluoromethoxyaniline.

MS (M⁺) m/z=349.

¹H NMR (DMSO-D₆, 300 MHz) δ 13.63 (broad s, 1H), 9.70 (broad s, 1H), 8.21 (d, 1H), 7.93 (d, 1H), 7.82 (dd, 1H), 7.49 (ddd, 1H), 6.83 (d, 1H).

### (b) Pyrazole-1,3-dicarboxylic acid 3-(2-bromo-4-trifluoromethoxyphenylamide) 1-(3-trifluoromethylphenylamide)

The title compound was prepared according to the procedure described above in Example 57(b) from 1H-pyrazole-3-carboxylic acid 2-bromo-4-trifluoromethoxyphenylamide (see step (a) above) and 3-trifluoromethylphenyl isocyanate.

MS (M⁺) m/z=536.

¹H NMR (DMSO-D₆, 300 MHz) δ 13.57 (s, 1H), 9.70 (s, 1H), 8.21 (d, 1H), 7.95 (d, 1H), 7.83 (dd, 1H), 7.54-7.47 (m, 1H), 7.20 (t, 1H), 6.86-6.73 (m, 3H).

### Example 69

### Pyrazole-1,3-dicarboxylic acid 1-(benzo[1,3]dioxol-5-ylamide) 3-(2-bromo-4-trifluoromethoxyphenylamide)

The title compound was prepared according to the procedure described above in Example 57(b) from 1H-pyrazole-3-carboxylic acid 2-bromo-4-trifluoromethoxyphenylamide (see Example 68(a)) and 3,4-(methylenedioxy)phenyl isocyanate.

MS (M⁺) m/z=512.

¹H NMR (CDCl₃, 300 MHz) δ 9.24 (s, 1H), 8.81 (s, 1H), 8.62 (d, 1H), 8.42 (d, 1H), 7.54 (dq, 1H), 7.34-7.29 (m, 2H), 7.09 (d, 1H), 6.94 (dd, 1H), 6.86 (d, 1H), 6.04 (s, 2H).

### Example 70

### Pyrazole-1,3-dicarboxylic acid 3-(2-bromo-4-trifluoromethoxyphenylamide) 1-pentylamide

The title compound was prepared according to the procedure described above in Example 57(b) from 1H-pyrazole-3-carboxylic acid 2-bromo-4-trifluoromethoxyphenylamide (see Example 68(a)) and pentyl isocyanate.

MS (M⁺) m/z=462.

¹H NMR (CDCl₃, 300 MHz) δ 9.22 (s, 1H), 8.61 (d, 1H), 8.33 (d, 1H), 7.52 (d, 1H), 7.29 (dd, 1H), 7.05 (t, 1H), 7.03 (t, 1H), 3.51 (q, 2H), 1.78-1.65 (m, 2H), 1.47-1.37 (m, 4H), 1.00-0.93 (m, 3H).

### Example 71

### 6-{[3-(2-Bromo-4-trifluoromethoxyphenylcarbamoyl)pyrazole-1-carbonyl]-amino}hexanoic acid ethyl ester

The title compound was prepared according to the procedure described above in Example 57(b) from 1H-pyrazole-3-carboxylic acid 2-bromo-4-trifluoromethoxyphenylamide (see Example 68(a)) and ethyl 6-isocyanatohexanoate.

MS (M⁺) m/z=534.

¹H NMR (CDCl₃, 300 MHz) δ 9.20 (s, 1H), 8.58 (d, 1H), 8.33 (d, 1H), 7.52 (dq, 1H), 7.29 (dd, 1H), 7.09 (t, 1H), 7.03 (d, 1H), 4.12 (q, 2H), 3.52 (q, 2H), 2.36 (t, 2H), 1.79-1.66 (m, 4H), 1.54-1.42 (m, 2H), 1.26 (t, 3H).

### Example 72

### 6-{[3-(3-Methylpyridin-2-ylcarbamoylpyrazole-1-carbonyl]amino}-hexanoic acid ethyl ester

### (a) 1H-Pyrazole-3-carboxylic acid (3-methylpyridin-2-yl)amide

The sub-title compound was prepared according to the procedure described above in Example 57(a) using dipyrazolo[1,5-a;1′,5′-d]pyrazine-4,9-dione (see Example 6(a)) and 2-amino-3-methylpyridine.

MS (M⁺) m/z=202.

¹H NMR (DMSO-D₆, 300 MHz) δ 13.44 (s, 1H), 10.09 (s, 1H), 8.29 (dd, 1H), 7.84 (s, 1H), 7.71 (dd, 1H), 7.24 (dd, 1H), 6.84 (s, 1H), 2.21 (s, 3H).

### (b) 6-{[3-(3-Methylpyridin-2-ylcarbamoyl)pyrazole-1-carbonyl]amino}hexanoic acid ethyl ester

The title compound was prepared according to the procedure described above in Example 57(b) from 1H-pyrazole-3-carboxylic acid (3-methylpyridin-2-yl)amide (see step (a) above) and ethyl 6-isocyanatohexanoate.

MS (M⁺) m/z=387.

¹H NMR (DMSO-D₆, 300 MHz) δ 10.30 (s, 1H), 8.69 (t, 1H), 8.39 (d, 1H), 8.33 (dd, 1H), 7.77 (dd, 1H), 7.30 (dd, 1H), 6.97 (d, 1H), 4.02 (q, 2H), 3.30 (dt, 2H), 2.29 (t, 2H), 2.23 (s, 3H), 1.64-1.50 (m, 4H), 1.40-1.27 (m, 2H), 1.15 (t, 3H).

### Example 73

### Pyrazole-1,3-dicarboxylic acid 3-(3-methylpyridin-2-yl)amide) 1-pentylamide

The title compound was prepared according to the procedure described above in Example 57(b) from 1H-pyrazole-3-carboxylic acid (3-methylpyridin-2-yl)amide (see Example 72(a)) and pentyl isocyanate.

MS(M⁺) m/z=315.

¹H NMR (CDCl₃, 300 MHz) δ 9.07 (s, 1H), 8.30 (d, 1H), 8.28 (d, 1H), 7.65 (d, 1H), 7.18 (dd, 1H), 7.12 (t, 1H), 6.98 (d, 1H), 3.44 (q, 2H), 2.38 (s, 3H), 1.71-1.59 (m, 2H), 1.43-1.33 (m, 4H), 0.97-0.89 (m, 3H).

### Example 74

### 6-{[3-(2-Methoxy-6-methylphenylcarbamoyl)pyrazole-1-carbonyl]amino}-hexanoic acid ethyl ester

### (a) 1H-Pyrazole-3-carboxylic acid 2-methoxy-6-methylphenylamide

The sub-title compound was prepared according to the procedure described above in Example 57(a) using dipyrazolo[1,5-a;1′,5′-d]pyrazine-4,9-dione (see Example 6(a)) and 2-methoxy-6-methylaniline.

MS (M⁺) m/z=231.

¹H NMR (DMSO-D₆, 300 MHz) δ 13.35 (s, 1H), 9.15 (s, 1H), 7.83 (s, 1H), 7.17 (dd, 1H), 6.89 (d, 1H), 6.85 (d, 1H), 6.75 (s, 1H), 3.73 (s, 3H), 2.15 (s, 3H).

### (b) 6-{[3-(2-Methoxy-6-methylphenylcarbamoyl)pyrazole-1-carbonyl]-amino}hexanoic acid ethyl ester

The title compound was prepared according to the procedure described above in Example 57(b) from 1H-pyrazole-3-carboxylic acid 2-methoxy-6-methylphenylamide (see step (a) above) and ethyl 6-isocyanatohexanoate.

MS (M⁺) m/z=416.

¹H NMR (CDCl₃, 300 MHz) δ 8.29 (d, 1H), 8.26 (s, 1H), 7.28 (s, 1H), 7.26-7.16 (m, 1H), 7.00 (d, 1H), 6.91 (d, 1H), 6.81 (d, 1H), 4.08 (q, 2H), 3.84 (s, 3H), 3.50 (q, 2H), 2.35 (t, 2H), 2.32 (s, 3H), 1.78-1.64 (m, 4H), 1.54-1.41 (m, 2H), 1.22 (t, 3H).

### Example 75

### Pyrazole-1,3-dicarboxylic acid 1-(benzo[1,3]dioxol-5-ylamide) 3-(4-trifluoromethylphenylamide)

### (a) 1H-Pyrazole-3-carboxylic acid 4-trifluoromethylphenylamide

The sub-title compound was prepared according to the procedure described above in Example 57(a) using dipyrazolo[1,5-a;1′,5′-d]pyrazine-4,9-dione (see Example 6(a)) and 4-trifluoromethylaniline.

MS (M⁺) m/z=255.

¹H NMR (DMSO-D₆, 300 MHz) δ 13.58 (s, 1H), 10.46 (s, 1H), 8.04 (d, 2H), 7.89 (d, 1H), 7.69 (d, 2H), 6.84 (d, 1H).

### (b) Pyrazole-1,3-dicarboxylic acid 1-(benzo[1,3]dioxol-5-ylamide) 3-(4-trifluoromethylphenylamide)

The title compound was prepared according to the procedure described above in Example 57(b) from 1H-pyrazole-3-carboxylic acid 4-trifluoromethyl-phenylamide (see step (a) above) and 3,4-(methylenedioxy)phenyl isocyanate.

MS (M⁺) m/z=418.

¹H NMR (CD₃CN, 300 MHz) δ 9.52 (s, 1H), 9.47 (s, 1H), 8.36 (d, 1H), 8.00 (d, 2H), 7.70 (d, 2H), 7.34 (d, 1H), 7.11 (dd, 1H), 6.99 (d, 1H), 6.88 (d, 1H), 6.01 (s, 2H).

### Example 76

### Pyrazole-1,3-dicarboxylic acid 1-pentylamide 3-(4-trifluoromethylphenylamide)

The title compound was prepared according to the procedure described above in Example 57(b) from 1H-pyrazole-3-carboxylic acid 4-trifluoromethylphenylamide (see Example 75(a)) and pentyl isocyanate.

MS (M⁺) m/z=368.

¹H NMR (CDCl₃, 300 MHz) δ 8.67 (s, 1H), 8.31 (d, 1H), 7.83 (d, 2H), 7.64 (d, 2H), 7.06 (t, 1H), 7.02 (d, 1H), 3.48 (q, 2H), 1.76-1.65 (m, 2H), 1.45-1.36 (m, 4H), 0.98-0.90 (m, 3H).

### Example 77

### 6-{[3-(4-Trifluoromethylphenylcarbamoyl)pyrazole-1-carbonyl]amino}hexanoic acid ethyl ester

The title compound was prepared according to the procedure described above in Example 57(b) from 1H-pyrazole-3-carboxylic acid 4-trifluoromethyl-phenylamide (see Example 75(a)) and ethyl 6-isocyanatohexanoate.

MS (M⁺) m/z=440.

¹H NMR (CDCl₃, 300 MHz) δ 8.86 (s, 1H), 8.31 (d, 1H), 7.87 (d, 2H), 7.65 (d, 2H), 7.19 (t, 1H), 7.03 (d, 1H), 4.10 (q, 2H), 3.52 (q, 2H), 2.38 (t, 2H), 1.80-1.68 (m, 4H), 1.55-1.43 (m, 2H), 1.23 (t, 3H).

### Example 78

### Pyrazole-1,3-dicarboxylic acid bis(benzo[1,3]dioxol-5-ylamide)

### (a) 1H-Pyrazole-3-carboxylic acid benzo[1,3]dioxol-5-ylamide

The sub-title compound was prepared according to the procedure described above in Example 57(a) using dipyrazolo[1,5-a;1′,5′-d]pyrazine-4,9-dione (see Example 6(a)) and 3,4-(methylenedioxy)aniline.

MS (M⁺) m/z=231.

¹H NMR (DMSO-D₆, 300 MHz) δ 13.39 (s, 1H), 9.97 (s, 1H), 7.88 (s, 1H), 7.47 (s, 1H), 7.27 (d, 1H), 6.87 (d, 1H), 6.74 (s, 1H), 5.99 (s, 2H).

### (b) Pyrazole-1,3-dicarboxylic acid bis(benzo[1,3]dioxol-5-ylamide)

The title compound was prepared according to the procedure described above in Example 57(b) from 1H-pyrazole-3-carboxylic acid benzo[1,3]dioxol-5-ylamide (see step (a) above) and 3,4-(methylenedioxy)phenyl isocyanate.

MS (M⁺) m/z=394.

¹H NMR (CD₃CN, 300 MHz) δ 9.42 (s, 1H), 9.18 (s, 1H), 8.37 (d, 1H), 7.47 (dd, 1H), 7.34 (dd, 1H), 7.17-7.07 (m, 2H), 6.97 (d, 1H), 6.93-6.85 (m, 2H), 6.02 (s, 2H), 6.00 (s, 2H).

### Example 79

### Pyrazole-1,3-dicarboxylic acid 3-(benzo[1,3]dioxol-5-ylamide) 1-pentylamide

The title compound was prepared according to the procedure described above in Example 57(b) from 1H-pyrazole-3-carboxylic acid benzo[1,3]dioxol-5-ylamide (see Example 78(a)) and pentyl isocyanate.

MS (M⁺) m/z=344.

¹H NMR (CDCl₃, 300 MHz) δ 8.46 (s, 1H), 8.27 (d, 1H), 7.39 (d, 1H), 7.08 (t, 1H), 6.98 (d, 1H), 6.95 (d, 1H), 6.78 (d, 1H), 5.98 (s, 2H), 3.46 (q, 2H), 1.74-1.63 (m, 2H), 1.43-1.35 (m, 4H), 0.97-0.89 (m, 3H).

### Example 80

### 6-{[3-(Benzo[1,3]dioxol-5-ylcarbamoyl)pyrazole-1-carbonyl]-amino}-hexanoic acid ethyl ester

The title compound was prepared according to the procedure described above in Example 57(b) from 1H-pyrazole-3-carboxylic acid benzo[1,3]dioxol-5-ylamide (see Example 78(a)) and ethyl 6-isocyanatohexanoate.

MS (M⁺) m/z=416.

¹H NMR (CDCl₃, 300 MHz) δ 8.63 (s, 1H), 8.27 (d, 1H), 7.40 (d, 1H), 7.22 (t, 1H), 6.99 (dd, 1H), 6.98 (d, 1H), 6.78 (d, 1H), 5.98 (s, 2H), 4.10 (q, 2H), 3.48 (q, 2H), 2.35 (t, 2H), 1.77-1.64 (m, 4H), 1.52-1.39 (m, 2H), 1.23 (t, 3H).

### Example 81

### Pyrazole-1,3-dicarboxylic acid 1-(benzo[1,3]dioxol-5-ylamide) 3-(3-chloro-4-fluorophenylamide)

### (a) 1H-Pyrazole-3-carboxylic acid 3-chloro-4-fluorophenylamide

The sub-title compound was prepared according to the procedure described above in Example 57(a) using dipyrazolo[1,5-a;1′,5′-d]pyrazine-4,9-dione (see Example 6(a)) and 3-chloro-4-fluoroaniline.

MS (M⁺) m/z=239.

¹H NMR (DMSO-D₆, 300 MHz) δ 13.60 (broad s, 1H), 10.30 (broad s, 1H), 8.09 (dd, 1H), 7.87 (s, 1H), 7.77 (ddd, 1H), 7.48 (t, 1H), 6.80 (s, 1H).

### (b) Pyrazole-1,3-dicarboxylic acid 1-(benzo[1,3]dioxol-5-ylamide) 3-(3-chloro-4-fluorophenylamide)

The title compound was prepared according to the procedure described above in Example 57(b) from 1H-pyrazole-3-carboxylic acid 3-chloro-4-fluorophenyl-amide (see step (a) above) and 3,4-(methylenedioxy)phenyl isocyanate.

MS (M⁺) m/z=402.

¹H NMR (CDCl₃, 300 MHz) δ 8.76 (s, 1H), 8.56 (s, 1H), 8.40 (d, 1H), 7.88 (dd, 1H), 7.58 (ddd, 1H), 7.29 (d, 1H), 7.18 (dd, 1H), 7.07 (d, 1H), 6.98 (dd, 1H), 6.85 (d, 1H), 6.04 (s, 2H).

### Example 82

### 6-{[3-(3-Chloro-4-fluorophenylcarbamoyl)pyrazole-1-carbonyl]amino}hexanoic acid ethyl ester

The title compound was prepared according to the procedure described above in Example 57(b) from 1H-pyrazole-3-carboxylic acid 3-chloro-4-fluorophenyl-amide (see Example 81(a)) and ethyl 6-isocyanatohexanoate.

MS (M⁺) m/z=424.

¹H NMR (CDCl₃, 300 MHz) δ 8.74 (s, 1H), 8.29 (d, 1H), 7.86 (dd, 1H), 7.58 (ddd, 1H), 7.19 (t, 1H), 7.14 (dd, 1H), 7.00 (d, 1H), 4.09 (q, 2H), 3.50 (q, 2H), 2.37 (t, 2H), 1.79-1.66 (m, 4H), 1.53-1.41 (m, 2H), 1.23 (t, 3H).

### Example 83

### Pyrazole-1,3-dicarboxylic acid 3-(3-chloro-4-fluorophenylamide) 1-pentylamide

The title compound was prepared according to the procedure described above in Example 57(b) from 1H-pyrazole-3-carboxylic acid 3-chloro-4-fluorophenyl-amide (see Example 81(a)) and pentyl isocyanate.

MS (M⁺) m/z=352.

¹H NMR (CDCl₃, 300 MHz) δ 8.53 (s, 1H), 8.30 (d, 1H), 7.83 (dd, 1H), 7.54 (ddd, 1H), 7.14 (dd, 1H), 7.07 (t, 1H), 6.99 (d, 1H), 3.47 (q, 2H), 1.75-1.62 (m, 2H), 1.45-1.34 (m, 4H), 0.98-0.89 (m, 3H).

### Example 84

The inhibition of 15-lipoxygenase obtained using the biological test method described above is exemplified by the compounds of the above examples, as listed in the following table:

Example 1: 45%

Example 8: 50%

Example 14: 39%

Example 23: 44%

Example 26: 45%

1. A compound of formula I, 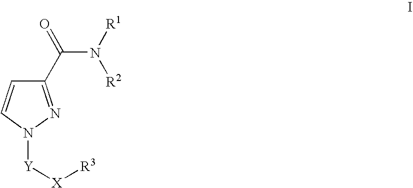
wherein either R¹ represents an aryl group or a heteroaryl group, both of which are optionally substituted by one or more substituents selected from G¹ and B¹, which B¹ group may itself be further substituted by one or more substituents selected from G², Z (provided that Z is not directly attached to an aryl or a heteroaryl group) and B² (which B² group is optionally further substituted by one or more substituents selected from G³, B³ and Z, provided that Z is not attached to an aryl or a heteroaryl group); and
R² represents H or C₁₋₆ alkyl, which latter group is optionally substituted by one or more halo groups; or
when R² represents C₁₋₆ alkyl optionally substituted by halo, R¹ and R² may be linked together forming a further 5- to 7-membered ring, optionally containing 1 to 3 heteroatoms and/or 1 to 3 double bonds, which ring is itself optionally substituted by one or more substituents selected from G¹, Z (provided that the ring is not aromatic in nature) and B¹ (which B¹ group is optionally substituted as described above);
R³ represents C₁₋₆ alkyl, C₂₋₆ alkenyl, C₂₋₆ alkynyl, C₃₋₈ cycloalkyl, C₃₋₈ heterocycloalkyl, aryl or heteroaryl, all of which groups are optionally substituted by one or more substituents selected from G^{1a}, Z (provided that Z is not directly attached to an aryl or a heteroaryl group) and B¹ (which B¹ group is optionally substituted as described above);
X represents a direct bond, —O— or —N(R⁴)—;
Y represents —C(O)—, —C(S)— or —S(O)₂—;
B¹, B² and B³ independently represent, on each occasion when used above, C₁₋₆ alkyl, C₂₋₆ alkenyl, C₂₋₆ alkynyl, C₃₋₈ cycloalkyl, C₃₋₈ heterocycloalkyl, aryl or heteroaryl;
G¹, G^{1a}, G² and G³ independently represent, on each occasion when used above, halo, cyano, —N₃, —NO₂, —ONO₂ or -A¹-R⁴;
wherein A¹ represents a spacer group selected from —C(Z)A²-, —N(R⁵)A³-, —OA⁴-, —S— or —S(O)ₙA⁵-, in which:
A² represents a single bond, —O—, —S— or —N(R⁵)—;
A³ represents A⁶, —C(Z)N(R⁵)C(Z)N(R⁵)—, —C(Z)N(R⁵)C(Z)O—, —C(Z)N(R⁵)S(O)ₙN(R⁵), —C(Z)S—, —S(O)ₙ—, —S(O)N(R)C(Z)N(R⁵)—, —S(O)N(R⁵)C(Z)O—, —S(O)ₙN(R⁵)S(O)ₙN(R⁵)—, —C(Z)O—, —S(O)ₙN(R⁵)— or —S(O)ₙO—;
A⁴ represents A⁶, —S(O)ₙ—, —C(Z)O—, —S(O)ₙN(R⁵)— or —S(O)ₙO—;
A⁵ represents a single bond, —N(R⁵)— or —O—;
A⁶ represents a single bond, —C(Z)- or —C(Z)N(R⁵)—;
Z represents, on each occasion when used above, a substituent connected by a double bond, which is selected from ═O, ═S, ═NR⁴, ═NN(R⁴)(R⁵), ═NOR⁴, ═NS(O)₂N(R⁴)(R⁵), ═NCN, ═CHNO₂ and ═C(R⁴)(R⁵);
R⁴ and R⁵ independently represent, on each occasion when used above, H or B⁴, which B⁴ group is itself optionally substituted by one or more substituents selected from G⁴, Q provided that Q is not directly attached to an aryl or a heteroaryl group) and B⁵ (which B⁵ group is itself optionally substituted by one or more substituents selected from G5, Q (provided that Q is not directly attached to an aryl or a heteroaryl group) and B⁶; or when R⁴ and R⁵ both represent optionally substituted B⁴ groups, then any pair thereof may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more substituents selected from G⁶, Q (provided that the ring is not aromatic in nature) and B⁴ (which B⁴ group is optionally substituted as described above);
B⁴, B⁵ and B⁶ independently represent on each occasion when used above C₁₋₆ alkyl, C₂₋₆ alkenyl, C₂₋₆ alkynyl, C₃₋₈ cycloalkyl, C₃₋₈ heterocycloalkyl, aryl or heteroaryl;
G⁴, G⁵ and G⁶ independently represent on each occasion when used above, halo, cyano, N₃, —NO₂, —ONO₂ or -A⁷-R⁶;
wherein A⁷ represents a spacer group selected from —C(Q)A⁸-, —N(R⁷)A⁹-, N(R^{7a})A^{9a}-, —OA¹⁰-, —S— or —S(O)ₙA¹¹-, in which:
A⁸ represents a single bond, —O—, —S— or —N(R⁷)—;
A⁹ represents A¹², —C(Q)S—, —S(O)ₙ—, —C(Q)O—, —S(O)ₙN(R⁷)— or —S(O)ₙO—;
A^{9a} represents —C(Q)N(R⁷)C(Q)N(R⁷)—, —C(Q)N(R⁷)C(Q)O—, —C(Q)N(R⁷)S(O)ₙN(R⁷)—, —S(O)ₙN(R⁷)C(Q)N(R⁷)—, —S(O)ₙN(R⁷)C(Q)O—, —S(O)ₙN(R⁷)S(O)ₙN(R⁷)_;
A¹⁰ represents A¹², —S(O)—, —C(Q)O—, —S(O)ₙN(R⁷)— or S(O)ₙO—;
A¹¹ represents a single bond, —N(R⁷)— or —O—;
A¹² represents a single bond, —C(Q)- or —C(Q)N(R⁷)—;
Q represents, on each occasion when used above, a substituent connected by a double bond, which is selected from ═O, ═S, ═NR⁶, ═NN(R⁶)(R⁷), ═NOR⁶, ═NS(O)₂N(R⁶)(R⁷), ═NCN, ═CHNO₂ and ═C(R⁶)(R⁷);
R⁶, R⁷ and R^{7a} independently represent, on each occasion when used above, H, C₁₋₆ alkyl, C₂₋₆ alkenyl, C₂₋₆ alkynyl, C₃₋₈ cycloalkyl, C₃₋₈, heterocycloalkyl, aryl or heteroaryl, which latter seven groups are optionally substituted by one or more groups selected from halo, C₁₋₆ alkyl (optionally substituted by one or more halo groups) —N(R⁸)R⁹, —OR⁸, —ONO₂ and —SR⁸; or
provided that they do not represent H, any pair of R⁶ and R⁷ may, for example when present on the same atom or on adjacent atoms, be linked together to form, with those, or other relevant, atoms, a 5- to 7-membered ring, optionally containing 1 to 3 heteroatoms and/or 1 to 3 double bonds, which ring is itself optionally substituted by one or more groups selected from halo, C₁₋₆ alkyl (optionally substituted by one or more halo groups), —N(R⁸)R⁹, —OR⁸, —ONO₂ and —SR⁸;
R⁸ and R⁹ independently represent, on each occasion when used above, H or C₁₋₄ alkyl, which latter group is optionally substituted by one or more halo groups; and n represents, on each occasion when used above, 1 or 2;
or a pharmaceutically-acceptable salt thereof,
provided that, when R² represents H, Y represents —C(O)— and:
(A) X represents a direct bond and:
i) R³ represents phenyl, then R¹ does not represent phenyl, 2-methoxyphenyl, 2-thiazolyl or 6-methyl-2-pyridinyl;
ii) R³ represents 4-fluorophenyl, then R¹ does not represent 2-carbomethoxyphenyl, 3-carbomethoxyphenyl or 2,4-dimethylphenyl;
iii) R³ represents 2-chlorophenyl, then R¹ does not represent phenyl, 3-bromophenyl or 4-bromophenyl;
iv) R³ represents 3-chlorophenyl, then R¹ does not represent phenyl, 2-fluorophenyl, 2-chlorophenyl, 2,3-dichlorophenyl or 2,5-dichlorophenyl;
v) R³ represents 4-chlorophenyl, then R¹ does not represent 3-bromophenyl or 4-methoxyphenyl;
vi) R³ represents 3-iodophenyl, then R¹ does not represent 2-methoxyphenyl or 2,4-dimethylphenyl;
vii) R³ represents 2,4-dichlororphenyl, then R¹ does not represent 4-chlorophenyl or 2,3-dichlorophenyl;
viii) R³ represents 3,5-dinitrophenyl, then R¹ does not represent 2,3-dichlorophenyl;
ix) R³ represents 2,4-dimethyl-6-oxo-6H-pyran-3-yl, then R¹ does not represent 3-carbomethoxyphenyl;
x) R³ represents methyl, then R¹ does not represent 3,4-dichlorophenyl, 2-methoxyphenyl, 2-thiazolyl, 4-methyl-2-pyridinyl, 6-methyl-2-pyridinyl or 4-acetylphenyl;
xi) R³ represents ethyl, then R¹ does not represent phenyl, 2,3-dichlorophenyl, 4-methoxyphenyl, 2-carbomethoxy-phenyl, 2-thiazolyl or 4-methyl-2-pyridinyl;
(B) X represents —N(H)— and:
i) R³ represents phenyl, then R¹ does not represent 4-methoxyphenyl, 2,4-dimethylphenyl or 2-thiazolyl;
ii) R³ represents 3-chlorophenyl, then R¹ does not represent 4-methylphenyl;
iii) R³ represents 4-chlorophenyl, then R¹ does not represent 3-bromophenyl;
iv) R³ represents 3,4-dichlorophenyl, then R¹ does not represent 4-methyl-2-pyridinyl or 6-methyl-2-pyridinyl;
v) R³ represents 2′-sulfamoylbiphenyl-4-yl, then R¹ does not represent 5-bromo-2-pyridinyl;
vi) R³ represents 1-propyl, then R¹ does not represent phenyl;
vii) R³ represents 1-butyl, then R¹ does not represent 4-bromophenyl or 2,4-dimethylphenyl;
viii) R³ represents cyclohexyl, then R¹ does not represent 4-methoxyphenyl;
(C) X represents —O— and:
i) R³ represents phenyl, then R¹ does not represent phenyl or 6-methyl-2-pyridinyl;
ii) R³ represents methyl, then R¹ does not represent phenyl, 2-fluorophenyl, 2,4-dimethylphenyl, 4-acetylphenyl or 2-thiazolyl;
iii) R³ represents ethyl, then R¹ does not represent phenyl, 2-fluorophenyl, 4-acetylphenyl or 4-methyl-2-pyridinyl;
iv) R³ represents 1-butyl, then R¹ does not represent 2-fluorophenyl, 2-methoxyphenyl, 4-methyl-2-pyridinyl or 6-methyl-2-pyridinyl;
v) R³ represents 2-butyl, then R¹ does not represent 2-thiazolyl or 4-acetylphenyl;
vi) R³ represents 2-methyl-1-propyl, then R¹ does not represent phenyl or 3-nitorphenyl.

2. A compound as claimed in claim 1, wherein R¹ represents an aryl or heteroaryl group, both of which are optionally substituted as defined in claim 1.

3. A compound as claimed in claim 1, wherein G¹ represents halo, cyano or -A¹-R⁴.

4. A compound as claimed in claim 1, wherein B¹ represents an optionally substituted C₁₋₆ alkyl, C₂₋₆ alkenyl, C₂₋₆ alkynyl, C₃₋₇ cycloalkyl, C₄₋₇ heterocycloalkyl, or phenyl, group.

5. A compound as claimed in claim 1, wherein G^{1a} represents halo, cyano, —NO₂ or -A¹-R⁴.

6. A compound as claimed in claim 1, wherein G² represents halo, cyano, —ONO₂ or -A¹-R⁴.

7. A compound as claimed in claim 1, wherein B² represents C₁₋₆ alkyl, C₂₋₆ alkenyl or C₂₋₆ alkynyl, all of which are optionally substituted by one or more G³ and/or B³ groups.

8. A compound as claimed in claim 1, wherein G³ represents halo, —ONO₂, —N(R⁵)(R⁴) or —OR⁴.

9. A compound as claimed in claim 1, wherein B³ represents C₁₋₆ alkyl, C₂₋₆ alkenyl or C₂₋₆ alkynyl.

10. A compound as claimed in claim 1, wherein when A¹ represents —N(R⁵)A³-, A³ represents A⁶, —C(Z)S—, —S(n)ₙ—, —C(Z)O— or —S(O)ₙN(R⁵)—.

11. A compound as claimed in claim 1, wherein when A¹ represents —OA⁴-, A⁴ represents A⁶.

12. A compound as claimed in claim 1, wherein when A¹ represents —S(O)ₙA⁵-, A⁵ represents a single bond or —N(R⁵)—.

13. A compound as claimed in claim 1, wherein when A¹ represents —C(Z)A²-, A² represents a single bond, —O— or —N(R⁵)—.

14. A compound as claimed in claim 1, wherein A¹ represents —C(Z)A²-, —N(R⁵)A³- or —OA⁴-.

15. A compound as claimed in claim 1, wherein Z represents ═O or ═NR⁴.

16. A compound as claimed in claim 1, wherein when any pair or R⁴ and R⁵ are linked together to form a ring, they are optionally substituted with G⁶ and/or B⁴.

17. A compound as claimed in claim 1, wherein G⁴ represents halo, cyano, —ONO₂ or -A⁷-R⁶.

18. A compound as claimed in claim 1, wherein B⁵ represents C₁₋₆ alkyl, C₂₋₄ alkenyl or C₂₋₆ alknyl, all of which are optionally substituted by one or more G¹ and/or B⁶ groups.

19. A compound as claimed in claim 1, wherein G⁵ represents halo, —ONO₂, —N(R⁷)(R⁶) or —OR⁶.

20. A compound as claimed in claim 1, wherein B⁶ represents C₁₋₆ alkyl, C₂₋₆ alkenyl or C₂₋₆ alkynyl.

21. A compound as claimed in claim 1, wherein G⁶ represents halo, cyano or -A⁷-R⁶.

22. A compound as claimed in claim 1, wherein A⁷ represents —C(Q)A⁸-, —N(R⁷)A⁹-, —OA¹⁰-, —S— or —S(O)ₙA¹¹-.

23. A compound as claimed in claim 1, wherein when A⁷ represents —N(R⁷)A⁹-, A⁹ represents A¹², —C(Q)S—, —S(O)ₙ—, —C(Q)O— or —S(O)ₙN(R⁷)—.

24. A compound as claimed in claim 1, wherein when A⁷ represents —OA¹⁰-, A¹⁰ represents A¹².

25. A compound as claimed in claim 1, wherein when A⁷ represents —S(O)ₙA¹¹-, A¹¹ represents a single bond or —N(R⁷)—.

26. A compound as claimed in claim 1, wherein when A⁷ represents —C(Q)A⁸-, A⁸ represents a single bond, —O— or —N(R⁷)—.

27. A compound as claimed in claim 1, wherein Q represents ═O or ═NR⁶.

28. A compound as claimed in claim 1, wherein R⁶, R⁷ and R^{7a} independently represent H, C₁₋₆ alkyl, C₂₋₆ alkenyl or C₂₋₆ alkynyl, all of which groups are optionally substituted by one or more groups selected from halo, C₁₋₆ alkyl, —N(R⁸)R⁹, OR⁸ and —ONO₂.

29. A compound as claimed in claim 1 wherein when any pair of R⁶ and R⁷ are linked together to form a ring, that ring is optionally substituted by one or more groups selected from halo, C₁₋₆ alkyl (optionally substituted by one or more halo groups), —N(R⁸)R⁹, —OR⁸ and —ONO₂.

30. A compound as claimed in claim 1, wherein B⁴ represents an optionally substituted C₁₋₆ alkyl, C₂₋₆ alkenyl, C₂₋₆ alkynyl, C₃₋₇ cycloalkyl, C₄₋₇ heterocycloalkyl, or phenyl, group.

31. A compound as claimed in claim 1 wherein R⁴ and/or R⁵ independently represent H or C₁₋₆ alkyl, which latter group is optionally substituted by one or more fluoro groups.

32. A compound as claimed in claim 1, wherein X represents a direct bond, —O—, —N(H)— or —N(Me)—.

33. A compound as claimed in claim 1 wherein R² represents H, methyl or ethyl.

34. A compound as claimed in claim 1, wherein R¹ represents an optionally substituted phenyl, naphthyl, pyrrolidinyl, piperidinyl, pyrrolyl, furanyl, thiophenyl, pyrazolyl, imidazolyl, oxazolyl, isoxazolyl, thiazolyl, pyridinyl, indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothiophenyl, pyridazinyl, pyrimidinyl, pyrazinyl, indazolyl, benzimidazolyl, quinazolinyl, quinoxalinyl, 1,3-benzodioxolyl, benzothiazolyl, or benzodioxanyl, group.

35. A compound as claimed in claim 34, wherein R¹ represents optionally substituted phenyl, 2-pyridinyl, 3-pyridinyl, 2-thiophenyl, 4-pyrazolyl, 5-isoxazolyl, 1,3-benzodioxolyl, indazolyl, benzothiazolyl, or quinolinyl, group.

36. A compound as claimed in claim 34, wherein the optional substituent(s) are selected from halo, cyano, C₁₋₆ alkyl (which alkyl group may be linear or branched, and/or substituted by one or more fluoro and/or C₃₋₆ cycloalkyl groups), C₂₋₆ alkenyl, C₃₋₆ cycloalkyl, phenyl, pyrrolidinyl, piperidinyl, piperazinyl, tetrahydrofuranyl, tetrahydropyranyl, morpholinyl, thiomethyl, methylsulfinyl, methylsulfonyl, —OR¹⁰, —N(R¹⁰)R¹¹, —C(Q)OR¹⁰, —C(Q)R¹⁰, —C(Q)N(R¹⁰)R¹¹, —S(O)₂N(R¹⁰)R¹¹ and —N(R¹⁰)S(O)₂R¹², wherein R¹⁰ and R¹¹ independently represent H, phenyl, C₁₋₆ alkyl (which alkyl group is optionally substituted by one or more fluoro atom), C₂₋₆ alkenyl or C₃₋₆ cycloalkyl; or R¹⁰ and R¹¹ may be linked together to form, with the nitrogen atom to which they are attached, a 5- to 7-membered ring, optionally containing one additional heteroatom and optionally substituted with one or more C₁₋₆ alkyl groups, which alkyl groups are themselves optionally substituted by one or more halo groups; and R¹² represents phenyl, C₁₋₆ alkyl (which alkyl group is optionally substituted by one or more fluoro atom), C₂₋₆ alkenyl or C₃₋₆ cycloalkyl.

37. A compound as claimed in claim 36, wherein the optional substituent(s) are selected from carbomethoxy, methyl, dimethylamino, cyano, cholor, fluoro, trifluoromethyl, bromo, methoxy and trifluoromethoxy.

38. A compound as claimed in claim 1, wherein R³ represents an optionally substituted C₁₋₆ alkyl, C₃₋₆ cycloalkyl, phenyl, naphthyl, pyrrolidinyl, piperidinyl, piperazinyl, pyrrolyl, furanyl, thiophenyl, pyrazolyl, imidazolyl, oxazolyl, isoxazolyl, thiazolyl, pyridinyl, indazolyl, indolyl, indolinyl, isoindolinyl, oxindolyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothiophenyl, pyridazinyl, pyrimidinyl, pyrazinyl, indazolyl, benzimidazolyl, quinazolinyl, quinoxalinyl, 1,3-benzodioxolyl, benzothiazolyl, or benzodioxanyl, group.

39. A compound as claimed in claim 38, wherein R³ represents an optionally substituted C₁₋₆ alkyl, cyclohexyl, phenyl, 2-thiophenyl, 2-furanyl, 3-furanyl, 2-pyrrolyl, 1-naphthyl, 4-piperazinyl, 4-piperidinyl, benzofuranyl, or 1,3-benzodioxolyl, group.

40. A compound as claimed in claim 38, wherein the optional substituent(s) are selected from halo, —NO₂, cyano, C₁₋₆ alkyl (which alkyl group may be linear or branched, and/or optionally substituted with one or more halo, C₁₋₆ alkyl, C₂₋₆ alkenyl and/or C₃₋₆ cycloalkyl, groups, which latter three groups are themselves optionally substituted with one or more halo and/or C₁₋₆ alkyl groups), C₂₋₆ alkenyl (optionally substituted with one or more C₁₋₆ alkyl groups), C₃₋₆ cycloalkyl (optionally substituted with one or more halo groups), phenyl (optionally substituted with one or more halo groups), pyrrolidinyl, piperidinyl, piperazinyl, tetrahydrofuranyl, tetrahydropyranyl, morpholinyl, thiomethyl, methylsufinyl, methylsulfonyl, ═O, —OR³, —N(R¹³)R¹⁴, —C(Q)OR¹³, —C(Q)R¹³, —C(Q)N(R¹³)R¹⁴, —S(O)₂N(R¹³)R¹⁴ and —N(R¹³)S(O)₂R¹⁵, wherein R¹³ and R¹⁴ independently represent H, phenyl, C₁₋₆ alkyl (which alkyl group is optionally substituted by one or more fluoro atom), C₂₋₆ alkenyl or C₃₋₆ cycloalkyl; or R¹³ and R¹⁴ may be linked together to form, with the nitrogen atom to which they are attached, a 5- to 7-membered ring, optionally containing one additional heteroatom and optionally substituted with one or more C₁₋₆ alkyl groups, which alkyl groups are themselves optionally substituted by one or more halo groups; and R¹⁵ represents phenyl, C₁₋₆ alkyl (which alkyl group is optionally substituted by one or more fluoro atom), C₂₋₆ alkenyl or C₃₋₄ cycloaklyl.

41. A compound as claimed in claim 40, wherein the optional substituent(s) are selected from methyl, ethyl, ethoxy, trifluoromethyl, fluoro, chloro, iodo, phenyl, 2-chlorophenyl, 4-chlorophenyl, n-pentyl, i-propyl, nitro, t-butyl, —CH₂CH═CHC₈H₁₇, trifluoroacetyl, carbomethoxy, carboethoxy and trifluoromethoxy.

42. A compound as claimed in claim 1, wherein R¹ is phenyl, 2-chlorophenyl, 2-chloro-4-fluorophenyl, 3-chloro-4-fluorophenyl, 2,6-dichlorophenyl, 5-chloro-2-cyanophenyl, 2-fluoro-5-trifluoromethylphenyl, 2-bromo-4-trifluoromethoxyphenyl, 2-methoxy-6-methylphenyl, 3-cyanophenyl, 4-trifluoromethylphenyl, 4-dimethylaminophenyl, 4-carbomethoxyphenyl, 1,3,5-trimethyl-1H-pyrazol-4-yl, 3-methylisoxazol-5-yl, 3-pyridinyl, 2-chloro-3-pyridinyl, 3-methyl-2-pyridinyl, 3-carbomethoxythiophen-2-yl or 1,3-benzodioxolyl;
R² is hydrogen or methyl;
R³ is methyl, n-butyl, n-pentyl, 1-octyl, oleoyl, (1R,2S,5R)-(−)-menthyl, 2-chlorobenzyl, benzyl, phenyl, 3-fluorophenyl, 3-chlorophenyl, 4-chlorophenyl, 2-fluoro-5-iodophenyl, 5-fluoro-2-methylphenyl, 4-tert-butyl-phenyl, 4-pentylphenyl, 3-trifluoromethylphenyl, 4-trifluoromethoxyphenyl, 4-nitrophenyl, 2-ethoxyphenyl, 1-naphthyl, 2-furanyl, 2,5-dimethyl-3-furanyl, 2-carbomethoxy-5-furanyl, 1-methyl-1H-pyrrol-2-yl, 3-methyl-2-benzofuranyl, 3-methyl-2-thiophenyl, 1(N)-methyl-4-piperazinyl, 1(N)-(2,2,2-trifluoroacetyl)piperidin-4-yl, ethylhexanoate or 1,3-benzodioxolyl; Y is —C(O)—, —C(S)— or —S(O)₂—; and
X is a bond, —N(H)—, —N(Me)—, or —O—.

43. (canceled)

44. A pharmaceutical formulation including a compound of formula I, as defined in claim 1, but without the provisos, or a pharmaceutically acceptable salt thereof, in admixture with a pharmaceutically acceptable adjuvant, diluent or carrier.

45. (canceled)

46. The method as claimed in claim 49 wherein the lipoxygenase is 15-lipoxygenase.

47. The method as claimed in claim 46, wherein the disease is inflammation and/or has an inflammatory component.

48. The method as claimed in claim 47 wherein the inflammatory disease is asthma, chronic obstructive pulmonary disease (COPD), pulmonary fibrosis, an allergic disorder, rhinitis, inflammatory bowel disease, an ulcer, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune disease, Alzheimer's disease, multiple sclerosis, sarcoidosis, Hodgkin's disease or another malignancy.

49. A method of treatment of a disease in which inhibition of the activity of a lipoxygenase is desired and/or required, which method comprises administration of a therapeutically effective amount of a compound of formula I as defined in claim 1, but without the provisos, or a pharmaceutically-acceptable salt thereof, to a patient suffering from, or susceptible to, such a condition.

50. A combination product comprising:
(A) a compound of formula I as defined in claim 1, but without the provisos; and
(B) another therapeutic agent that is useful in the treatment of inflammation, wherein each of components (A) and (B) is formulated in admixture with a pharmaceutically-acceptable adjuvant, diluent or carrier.

51. A combination product as claimed in claim 50 which comprises a pharmaceutical formulation including the compound of formula I, another therapeutic agent that is useful in the treatment of inflammation, and a pharmaceutically-acceptable adjuvant, diluent or carrier.

52. A combination product as claimed in claim 50 which comprises a kit of parts comprising components:
(a) a pharmaceutical formulation including the compound of formula I, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

53. A process for the preparation of a compound as defined in claim 1, which comprises:
(i) for compounds of formula I in which, when Y is —S(O)₂—, X represents a direct bond or —N(R⁴)—, in which R⁴ represents B⁴, reaction of a compound of formula II, 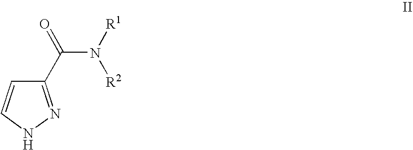
wherein R¹ and R² are as defined in claim 1, with a compound of formula III,
R³—X^{a}—Y-L¹ III
wherein X⁸ represents a direct bond or —N(B⁴)— when Y represents —S(O)₂— or, for all other values of Y, represents X as defined in claim 1, R³ and Y are as defined in claim 1 and L¹ represents a suitable leaving group;
(ii) for compounds of formula I in which X represents a single bond and Y represents —C(O)—, reaction of a compound of formula II as defined above with a compound of formula IV,
R³C(O)OH IV
wherein R³ is as defined in claim 1;
(iii) for compounds of formula I in which X represents a direct bond and Y represents a —C(O)— or a —C(S)— group, reaction of a compound of formula II as defined above with a compound of formula V,
R³═Y^{a} V
wherein Y⁸ represents —C(O)— or —C(S)— and R³ is as defined in claim 1;
(iv) for compounds of formula I, in which X represents —NH— and Y represents —C(O)— or —C(S)—, reaction of a compound of formula II as defined above with a compound of formula VI,
R³N═Y^{a} VI
wherein R³ is as defined in claim 1 and Y^{a} is as defined above;
(v) for compounds of formula I in which Y represents —C(O)— or —C(S)—, reaction of a compound of formula II as defined above with:
(a) a compound of formula VII,
Cl—Y^{a}—Cl VII
(b) a compound of formula VIII, 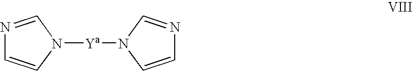
wherein, in both cases, Y^{a}8 is as defined above; or
(c) when Y represents —C(O)—, triphosgene, followed by:
(1) for compounds of formula I in which X represents a direct bond, reaction with a compounds of formula IX,
R³M IX
wherein M represents a metal such as Mn, Fe, Ni, Cu, Zn, Pd or Ce, or a salt or complex thereof and R³ is as defined in claim 1;
(2) for compounds of formula I wherein X represents O, reaction with a compound of formula X,
R³OH X
wherein R³ is as defined in claim 1; or
(3) for compounds of formula I wherein X represents —N(R⁴)—, reaction with a compound of formula X¹,
R³N(H)R⁴ XI
wherein R³ and R⁴ are as defined in claim 1;
(vi) for compounds of formula I in which X represents —N(R⁴)— and R⁴ is other than hydrogen, reaction of a corresponding compound of formula I in which X represents —N(H)— with a compound of formula XII,
R⁴-L¹ XII
wherein R⁴ is as defined in claim 1 and L¹ is as defined above;
(vii) for compounds of formula I in which Y represents —C(S)—, reaction of a corresponding compound of formula I in which Y represents —C(O)— with a suitable reagent for the conversion of a carbonyl group to a thiocarbonyl group;
(viii) reaction of a compound of formula XIII, 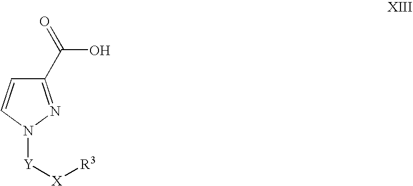
wherein R³, Y and X are defined in claim 1, with a compound of formula XIV,
HN(R¹)(R²) XIV
wherein R¹ and R² are as defined in claim 1; or
(ix) reaction of a compound of formula XV, 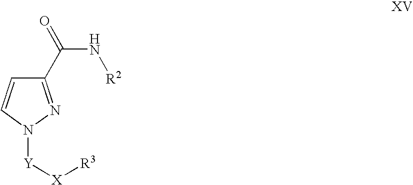
wherein R², R³, Y and X are as defined in claim 1, with a compound of formula XVI,
R¹-L² XVI
wherein L² represents a suitable leaving group and R¹ is as defined in claim 1.
